(19) United States
(12) Patent Application Publication
(10) Pub. No.: US 2011/0105869 A1
(43) Pub. Date: May 5, 2011

(54) Sensor for Internal Monitoring of Tissue O2 and/or pH/CO2 In Vivo

(75) Inventors: David F. Wilson, Philadelphia, PA (US); Sergei Vinogradov, Wynnwood, PA (US)

(73) Assignee: The Trustees of the University of Pennsylvania, Philadelphia, PA (US)

(21) Appl. No.: 12/942,696

(22) Filed: Nov. 9, 2010

Related U.S. Application Data
(63) Continuation-in-part of application No. 12/087,391, filed on Nov. 12, 2008.
(60) Provisional application No. 60/756,112, filed on Jan. 4, 2006.
(60) Provisional application No. 61/259,310, filed on Nov. 9, 2009.

(51) Int. Cl. A61B 5/1455

(52) U.S. Cl. 600/323

(57) ABSTRACT

Provided is a durable tissue pH/pCO₂ and/or tissue oxygen sensitive probe of sufficient strength to withstand direct tissue pressures in vivo, the probe comprising one or more sensor chambers within a biocompatible, gas-permeable membrane containing together in a single chamber, or in separate chambers, respectively, a pH sensitive fluorophor from which pCO₂ level(s) are calculated when the fluorophor is excited and the resulting fluorescence is measured and/or an oxygen sensitive phosphor solution producing oxygen quenchable phosphorescence when excited. Further provided is a tissue pH/pCO₂ and/or tissue oxygen detection and measurement system comprising the probe, and methods for use of the probe and the system to directly, rapidly and accurately measure tissue pH/pCO₂ and/or tissue oxygen levels in a patient without reliance on blood vessels or fluid protection of the probe.

### REFERENCE TO RELATED APPLICATIONS

This patent application is a Continuation-in-Part of U.S. patent application Ser. No. 12/087,391, filed Jul. 3, 2008, which claims priority to Provisional Application 60/756,112, filed Jan. 4, 2006 and PCT Application PCT/US2007/000292, filed Jan. 4, 2007, and to Provisional Application 61/259,310, filed Nov. 9, 2009, each of which is herein incorporated in its entirety.

### GOVERNMENT INTEREST

This invention was supported in part by Grant No. 5R01HL081273 from the U.S. National Institutes of Health. The U.S. Government may therefore have certain rights to use this invention.

### FIELD OF THE INVENTION

The present invention provides a sensor for measuring peripheral oxygen (pO₂) and one for peripheral CO₂ (pCO₂), and/or the two combined, and temperature in tissue in vivo in real-time, and methods of use thereof. This technology will be particularly important for patients with acute hemorrhage, those who have had heart attacks, those entering shock, as well as for those charged with caring for such patients, especially hospitals, nursing facilities, and first responders in both civilian and military settings.

### BACKGROUND OF THE INVENTION

The present invention is based upon the phenomenon that tissue oxygenation impacts a wide range of medical pathologies and patient outcomes. Tissue O₂ and CO₂ levels are very sensitive measures of metabolism and they change as a patient begins to lose cardiopulmonary efficiency. It is generally believed that peripheral hypoperfusion is a marker for worsening patient condition, because it coincides with the body's shunting of blood to the internal organs and away from the periphery (Finch and Lenfant, 1972). When blood flow (oxygen delivery) to the tissue is insufficient to meet the metabolic needs, there is typically also a deficiency in the removal of carbon dioxide, a metabolic product, and in peripheral tissue there may be a decrease in internal temperature of the tissue. Additionally, pathologies that decrease tissue oxygen levels through decreased oxygen delivery will typically cause an increase in pCO₂. This is important because insufficient oxygen and increased pCO₂ levels impair cell metabolism and decrease vascular resistance. This results in an increased work load for the heart, and as more tissue is affected, the load on the cardiovascular system increases. When there is also low blood volume (hemorrhage) or large amounts of tissue are affected (endotoxin, widespread infections) there may be progressive cardiovascular failure leading to multi-organ failure. As a result, improving outcomes from traumatic injury, for example, depends largely on earlier recognition of progression toward shock and on earlier intervention.

Currently clinicians often are not aware of the seriousness of the patient's condition until the organs begin to fail, and it is only then that they manage the consequences. Measuring these two critical physiological parameters can be difficult or misleading by present methods because they tend to respond differently depending on the type of pathology involved. This substantially increases the sensitivity to the stimulus and the reliability with which the data can be interpreted. For example, decreased blood flow (ischemia), such as occurs when the cardiovascular system is not functioning properly, results in decreased tissue pO₂ and increased pCO₂; whereas if the pulmonary function is compromised (hypoxia) the pO₂ decreases but there is much less change in the pCO₂ in the tissue.

At the present, the standard of care for measuring peripheral pO₂ and/or pCO₂ involves drawing blood from the patient and running the sample through a blood gas analyzer. The analyzer then takes several minutes to determine the gases present. Additionally, temperature and gas levels in the tissue can be substantially different than levels in the blood. As a result, there has been an unmet need for a way to measure real-time critical metabolic parameters that occur when a patient is suffering from a heart attack, hemorrhaging, or entering shock. This would allows for real-time results to be continuously read and the data to be streamed via wireless technology to a monitor, particularly if the system is durable, portable, and small enough to be carried by emergency medical personnel anywhere.

Fiber-optic sensors have been used to measure oxygen levels in vivo by positioning an analyte-sensitive indicator molecule in a light path at a desired measurement site. Typically, the optical fiber transmits electromagnetic radiation from a light source to the indicator molecule, and the reflectance from or absorption of light by the indicator molecule gives an indication of the gaseous or ionic concentration of the analyte. Alternatively, for monitoring an analyte, such as oxygen, the optical fiber transmits electromagnetic radiation to the indicator molecule, exciting it into a type of luminescence, i.e., phosphorescence, and the level and/or duration of phosphorescence by the indicator molecule serves as an indication of the concentration of the gas in the surrounding fluid. In the prior art sensors, the indicator molecules are typically disposed in a sealed chamber at the distal end of an optical fiber, and the chamber walls are permeable to the analytes of interest.

Several sensor devices are known which are useful for measuring oxygen and pH content in human and animal tissues by insertion of a light-sensing, optical fiber probe into a blood vessel of the subject. See, for example, U.S. Pat. No. 5,830,138 providing a detection device for measuring tissue oxygen and/or pH (CO₂) via insertion of a probe into a blood vessel of a subject in vivo, wherein the probe comprises a fiber optic means enclosed within a gas-permeable film. Situated between the gas-permeable film and the fiber optic means is a reservoir of a liquid, containing an aqueous oxygen-quenchable, phosphorescence-emitting oxygen sensor and/or a fluorescence-emitting pH sensor, and further comprising a means for detecting phosphorescent and/or fluorescent excitation light.

U.S. Pat. No. 4,758,814 provides an optical fiber covered by a membrane constructed of a hydrophilic porous material containing a pH sensitive dye for measuring blood pH levels, and having embedded in the membrane several hydrophobic microspheres containing a fluorescent dye quenchable by oxygen to simultaneously or sequentially measure oxygen partial pressure. Another fluorometric oxygen sensing device is described in U.S. Pat. No. 5,012,809, wherein the fluorometric sensor is constructed with silicone polycarbonate bonded to one or more plastic fiber optic light pipes using polymethylmethacrylate glues. U.S. Pat. No. 5,127,405 provides another version of a fiber optic probe containing an oxygen-permeable transport resin embedded with a luminescent composition comprising crystals of an oxygen quenchable phosphorescent material, whereby frequency domain representations are used to derive values for luminescence lifetimes or decay parameters. U.S. Pat. No. 4,752,115 employs an optical fiber, 250 nm in diameter or small enough for insertion into veins and/or arteries, wherein the probe is coated with an oxygen sensitive (oxygen quenchable) fluorescent dye which fluoresces light back to measure regional oxygen partial pressure, and wherein the oxygen sensing end of the probe may further include a gas-permeable sleeve over the optical fiber.

U.S. Pat. No. 4,476,870 discloses a fiber optic probe for implantation in the human body for gaseous oxygen measurement in the blood stream by means of a probe employing oxygen-quenchable, fluorescent dye enveloped in a hydrophobic, gas-permeable material at the end of two 150 um strands of a plastic optical fiber. U.S. Pat. No. 4,200,110 discloses a fiber optic pH probe employing an ion-permeable membrane envelope enclosing the ends of a pair of optical fibers, with a pH sensitive dye indicator composition disposed within the envelope. U.S. Pat. Nos. 3,814,081 and 3,787,119 describe early versions of such probes using photosensitive cells to determine physical and chemical characteristics of blood in vivo by direct measurement of light, but without oxygen quenchable phosphor/fluorophor compounds.

However, while the prior art probes are intended for measuring “tissue oxygen” in a patient in vivo, they require insertion into the lumen of a blood vessel and actually measure blood gases, not oxygen in the tissue surrounding the vessel. Blood flow rapidly changes the oxygen level within a given point in the vessel and would offer no way of measuring tissue oxygen in, for example, necrosing tissue. Moreover, the prior art systems cannot be effective unless the regions are well supplied with large vessels, such as muscle tissue, or in damaged tissue areas where the blood vessels are no longer intact, as in emergency situations.

One structural problem with the prior art sensing systems of the type described for use in blood vessels, is that the structure of the chambers and probe configuration often encourage the formation of blood clots or thrombi. Particularly when multiple fibers are used to determine several blood gas parameters, such as oxygen, carbon dioxide, and pH together, the probe provides interfiber crevices that encourage thrombi formation. Furthermore, the complexity and difficulty of manufacturing multi-fiber probes is well known, due to the small diameters of the fibers and requirements of their arrangement. Such probes must be small enough to fit within a blood vessel while still permitting blood to flow, especially problematic for neonatal or pediatric applications in which the patient's veins or arteries may be too small in diameter for insertion of the probe assembly.

In the hands of technically skilled and thoroughly knowledgeable investigators the prior art sensors are excellent research tools, but it is difficult to construct really good small electrodes. Calibration must be regularly checked. To avoid compression artifacts and errors due to tissue damage by the electrode, elaborate insertion protocols have been used, with a quick insertion step followed by a smaller withdrawal step and making the measurements very quickly (see Baumgärtl et al., Comp. Biochem. & Physiol. Part A 132: 75-85 (2002)). Recently, optical sensors similar to oxygen electrodes have used optical fibers coated with plastic containing oxygen sensitive dyes for measuring oxygen. These suffer from many of the same problems as oxygen electrodes, errors due to tissue compression and tissue damage, empirical calibration, poor long term stability, and exposure of the tissue to plastic that has included oxygen sensitive dye, and therefore, needs to be medically tested and approved.

Moreover, correctly placing the sensing end of the probe in the blood vessel and maintaining that placement for continued monitoring is important for obtaining reliable blood gas results. The prior art tissue oxygen or multi-analyte sensors have failed to effectively deal with the problems set forth above, and none offer a method for measuring oxygen in tissue other than via a blood vessel.

The design of the prior art probes is distinctly different from a device that can directly measure analyte levels in tissue, although similar sensor compositions and detection monitors may be used. A tissue probes that is not protected by a blood vessel, must withstand much higher local tissue pressures. For example, if prior art probes were inserted directly into tissue, rather than into a blood vessel, they would collapse or be disabled under the pressure of the surrounding tissue. They lack sufficient wall strength to withstand tissue pressure without the protection of a blood vessel and a surrounding fluid environment. Consequently, without the protection by the blood and blood vessel, insertion of a prior art probe directly into a non-fluid, tissue environment could compress and damage the sensor chamber, resulting in failure or a significantly decreased excitation of a phosphor sensor, as well as decreased collection of the returned phosphorescent excitation light. Side pressures could further cause sharp bends or “kinks” immediately adjacent to the optical fibers, which must be accounted for in the probe design.

Thus, until the present invention there has remained a need in the art to provide an improved device and method for directly, rapidly and accurately measuring real-time measurement of tissue oxygen and/or pCO₂ and temperature in a patient in vivo. As such, the sensor is of such a small size, enabling it to be inserted into the peripheral tissue of the patient, including but not limited to muscle and/or fatty tissue, with care taken to avoid insertion into a blood vessel, as vascular insertion results in a measurement of the blood gas levels as opposed to the desired measurement of tissue gas levels. Thus, the data from the device, sent via a wireless transmitter to a monitor, as provided by the present invention advantageously offers continuous, real-time measurement of tissue oxygen and/or pCO₂ and temperature in peripheral tissue, helping to reduce multi-organ failure via earlier patient management. Moreover, such information is also highly beneficial as a diagnostic tool, and will facilitate the quick, accurate and precise identification of many otherwise difficult-to-diagnose maladies or detecting life-threatening situations.

### SUMMARY OF THE INVENTION

In accordance with the present invention, tissue oxygen and/or pH/pCO₂ levels in tissue take advantage of novel phosphorescence emitting (“phosphors”) and/or fluorescence emitting compounds (“fluorophores”). Probes containing the sensor of the present invention are distinguished from the prior art in that it is place directly into the patient's tissue; it is not delivered into the body via the lumen of a blood vessel. As a result, the device, system and methods of the present invention directly measure tissue oxygen and/or pH/pCO₂ in the capillary bed of the selected tissue; this is not a measure of blood gases within a vessel. In use, the probe of the present invention is not protected by the blood vessel and surrounding fluids, thus the design is necessarily different from prior art technologies that operate from within a blood vessel. In accordance with the present invention, the tissue oxygen levels and/or the pH/pCO₂ levels may be read directly.

The unique design and placement within the tissue, particularly effective within muscle tissue, permits the pO₂ and/or pH/pCO₂ probe to be rapidly inserted in a matter of seconds, even under difficult conditions, such as those often faced by first medical responders. Once in place, the probe provides immediate data regarding cardiac and pulmonary function (tissue pO₂ and/or pH/pCO₂) to facilitate rapid and accurate treatment of the patient.

Thus, the present invention provides a device and system for detecting and directly measuring pO₂ and/or pH/pCO₂ in tissue of a patient (without reliance on an adjacent blood vessel or fluid environment), wherein the device comprises a sensor chamber B enclosed within a gas-permeable layer 2, the sensor containing an analyte solution comprising an aqueously-soluble, phosphor and/or fluorophor 3, respectively, wherein refractive index of the analyte solution in the sensor chamber is higher than that of the surrounding gas permeable layer 2; a light source for transmitting controlled excitation light to the analyte; and a detecting device for detecting light emitted from the excited analyte. The component comprising the sensor chamber and analyte within a gas permeable layer 2 are referred to as the “tissue probe” A. The tissue probe device combined with the excitation and detection means form the “system” of the invention. The volume of the tissue area that can be analyzed by a probe is typically a three-dimensional region measuring at least about 5-10 mm on a side.

It is a further object of the invention to provide a tissue probe A for use in the system as described, which is effectively used directly in the tissue of a patient without requiring the protection and limitations of insertion into the patient via a blood vessel. In one aspect of the invention, the probe comprises a fluorophor (also referred to herein as the “analyte”) dissolved in solution in an aqueous solvent within the sensor chamber B. In contrast to the prior art in this area, when a probe is used in a vein or artery, it must be less than 200-300 μm in diameter to permit passage into the lumen of the vessel. In contrast, the present invention is not so limited, providing distinct advantages over intravascular prior art devices.

In yet another embodiment of the invention, it is a further object to provide a phosphorescent analyte (a “phosphor”) within the sensor chamber of the tissue probe to detect and measure O₂ real-time in the patient tissue as described in co-owned U.S. patent application Ser. No. 12/087,391. In an alternative embodiment of the invention, it is a further object to provide a fluorescent analyte (a “fluorophor”) in solution, such as in bicarbonate solution) within the sensor chamber of the tissue probe to detect and measure pCO₂ real-time in the patient tissue. Such embodiments may also be combined with or used in addition to the fluorophor and/or phosphor sensor, respectively, of the present invention, but would be activated and measured in the manner described herein. In addition, probes to measure temperature and/or K⁺ ion levels of the tissue may also be included with the gas analyte(s).

It is also an object of the invention to provide a system comprising the probe, operably attached to one or more optic fibers 4 and 5 having two opposing ends. For discussion purposes, the device of the present invention embodied with optic fibers for transmitting light has a proximal end and a distal end. The distal end of the device comprises the probe containing the sensor chamber that is inserted into the patient's tissue in accordance with recognized medical practices. The distal end(s) of the fiber(s) are connected to, and form, a water-tight and durable seal with the probe. These distal end(s) of the optical fiber(s) are further enclosed within a tube of a gas-permeable layer 2 extending from the layer enclosing the sensor, thereby forming a light guide. The phosphorescence and/or fluorescence, provided when the phosphor and/or fluorophor, respectively, is excited, has substantially the same refractive index as the optical fibers.

At least one of the fibers transmits excitation light from an external light source at the proximal end of the optical fiber(s) to the fluorescent analyte. Conversely, at least one fiber collects emitted light from the analyte and transmits the collected, emitted light to an external detector device, which is also connected to the proximal end of the fiber(s). Thus, at the proximal end of the optical fiber(s) are the light source and detection components of the system external to the point of entry into the patient or extending externally beyond the point of entry.

Further provided are embodiments wherein the probe is inserted into the patient's tissue as described, but the excitation light is provided transdermally from outside of the patient to the probe without an optical fiber connection. Similarly, the collection and detection of the phosphorescence can be conducted transdermally from outside of the patient without an optical fiber connection.

Further, in accordance with the invention, light-emitting diodes are used for excitation of the fluorescence and/or phosphorescence, thereby taking advantage of their ability to provide a bright monochromatic light source which can easily be modulated at the required frequency and with the desired waveform.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

FIG. 1 depicts an embodiment of the invention showing a side-view cross-section of the sensor end of the probe showing a single optical fiber for transmitting excitation light and one for collecting emitted phosphorescence.

FIG. 2 depicts an embodiment of the invention showing a side-view cross-section of the sensor end of the tube showing multiple optical fibers for transmitting excitation light.

FIG. 3 depicts an embodiment of the invention showing a side-view cross-section of the system including the probe of FIG. 1 attached to the excitation and detection devices by one or more optical fibers, and showing the retractable needle 7 in its retracted position. The drawing is not to scale and as shown by the cuts in the optical fibers, they can be of any length

FIG. 4 graphically depicts fluorescence intensity ratios I₆₃₀/I₇₀₃ versus pH in H₂PorphGlu^{N}-OH(N=1-3) series. The emission spectra are obtained from excitation at 485 nm. Lines show analytical fitting of the data to Henderson-Hasselbalch curves with N=1, corresponding to the first N-protonation (K₃).

### DETAILED DESCRIPTION OF CERTAIN EMBODIMENTS OF THE INVENTION

The present invention comprises a system and device for directly detecting and measuring tissue oxygenation, specifically pO₂ and/or pH/pCO₂, without using a blood vessel to deliver the sensor probe to the region of interest. The system for measuring pO₂ and/or pCO₂ comprises a fiber optic sensor chamber B, at least one excitation light source for phosphorescence and/or fluorescence measurement, respectively, at least one combiner for coupling the laser diodes and photodiode to the optical fiber leading to the sensor chamber B, and at least one instrument with a central processor for measuring the resulting phosphorescence and/or fluorescence from the sensor chamber. In certain embodiments, a temperature sensor circuit is present.

In one embodiment of the invention, the fiber optic sensor chamber comprises a highly fluorescent pH indicator in a buffer (e.g., bicarbonate) which can be used in ratiometric mode to measure pH. In addition to the chamber, the sensor device comprises a small piece of tubing, and at least one optical fiber. The pH indicator is preferably aqueously soluble, has a pK between 5 and 7.5, with a preferred pK of 6.5, and has no known toxicity for biological materials. Such a pH indicator is desirable because the relationship of pH to the CO₂ pressure in buffers (e.g., bicarbonate) is well known and the system makes use of this relationship to measure pCO₂. Any pH may be used within the range, including 5.1, 5.2, 5.3, 5.4, 5.5, 5.6, 5.7, 5.8, 5.9, 6.1, 6.2, 6.3, 6.4, 6.5, 6.6, 6.7, 6.8, 6.9, 7.1, 7.2, 7.3, 7.4 or 7.5, and more narrow ranges within those pH designations.

Carbon dioxide is a weak acid and when it is dissolved in water or aqueous solution, it undergoes the following reactions:

CO₂+H₂O←→H₂CO₃←→H⁺+HCO₃⁻←→H⁺+CO₃⁻² (Equation 1)

The equilibrium expression for the first two steps of the equilibrium involves the molecular species primarily present in physiological conditions. The pK for the dissociation of the second proton (bicarbonate-carbonate) is very alkaline (approx. 9.5), whereas that for the dissociation of the first proton to form bicarbonate is at or near 6.1. The latter is the part used to measure the pCO₂ in tissue.

Keq2=[HCO₃⁻][H⁺]/[H₂CO₃] or Keq2=[HCO₃⁻]/[H₂CO₃]×[H⁺] (Equation 2)

Keq1=[H₂CO₃]/[H₂O]pCO₂ or Keq1=[H₂CO₃]/[H₂O]×pCO₂ (Equation 3)

The first two reactions are combined by solving Equation 3 for [H₂CO₃] and substituting into Equation 2.

[H₂CO₃]=Keq1/[H₂O]pCO₂ (Equation 4)

or

Keq2=[HCO₃⁻][H⁺]/Keq1/[H₂O]pCO₂ (Equation 5)

Keq2 Keq1=K=[HCO₃⁻][H⁺]/[H₂O]pCO₂ (Equation 6)

pCO₂=[HCO₃⁻][H⁺]/K[H₂O]=[HCO₃⁻]/K[H₂O]+[H⁺] (Equation 7)

By convention, the solvent has an activity of 1 and the equation becomes:

pCO₂/[HCO₃⁻]=1/K+[H⁺] or Log {pCO₂/[HCO₃⁻]}=pK−pH (Equation 8)

Therefore, the relationship of pCO₂ and pH is:

pH=pK−Log {pCO₂/[HCO₃⁻]} (Equation 9)

As such, if the medium within the sensor chamber contains a known concentration of bicarbonate, then pCO₂ can be accurately calculated from the measured pH in real-time.

The pH indicator dyes of choice are recently developed pH sensors based on porphyrin cores placed in appropriate molecular environments. Exemplary pH sensors are described in “Precise detection of pH inside large unilamellar vesicles using membrane-impermeable dendritic porphyrin-based nanoprobes” by Leiding et al., Anal. Biochem. 388:296-305 (2009), incorporated herein in its entirety. These dyes have very strong absorption, high quantum efficiency for fluorescence, appropriate pK values (6.1 to 6.5) and the emission peaks for the acid and alkaline forms and have quite different wavelengths. This allows ratiometric pH measurements, which are important because the ratio is measured accurately, independent of the absolute intensities of the signals and the changes in the ratio are much larger than for the fluorescence intensity. Further, measurements of pCO₂ based on this method do not require on site calibration.

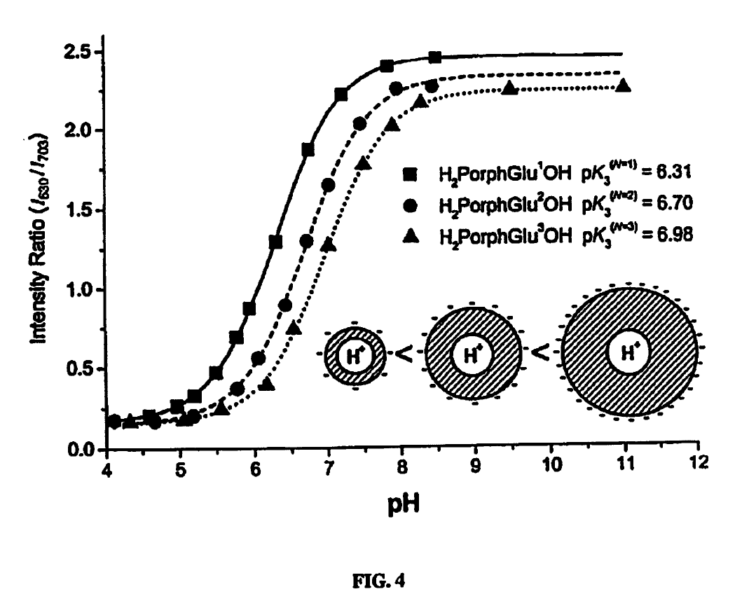

FIG. 4 shows that porphyrin based dyes are sensitive to pH in the appropriate range, but the pK is very sensitive to the ionic strength and ionic content of the surrounding medium. Thus, these dyes are not good pH indicators for most applications. Nevertheless, their use in the present invention is a special and unexpected case, wherein the indicator is in a solution that is completely determined (i.e., there is no exchange of ions between the surrounding medium and those that are inside the sensor chamber). Under these conditions these porphyrin-based dyes are excellent pH indicators with very high sensitivity, high absorption coefficients for excitation, and ideal pK for measuring pCO₂. Although photo-destruction of the pH indicator may eventually limit the long-term stability of the sensor, sensor chambers with the porphyrin-based pH sensors are designed to function effectively for at least 24 hours (including 1-24 hours), up to several days (1, 2, 3, 4 or 5 days or more) of continuous measurements in the patient's tissue. The analyte remains stabile in the sensor chamber B, and isolated from direct contact with the tissue, until the sensor is removed.

In one embodiment, a solution containing the pH indicator at a concentration of 0.1 to 100 micromolar is contained within the fiber optic sensor chamber (including 0.1, 0.2, and numerical increments up to 100 micromolar, specifically 5, 10, 20, 30, 40, 50, 60, 70, 80, 90 micromolar, and ranges within those concentrations). Optimal concentrations of pH indicators vary, depending on the efficiency of the optics, the intensity of the excitation source, and the sensitivity of the detector used to measure the fluorescence.

In another embodiment, the fiber optic sensor chamber B comprises water soluble, oxygen sensitive phosphor (also referred to, for the purposes of this invention, as an “oxyphor”) in solution, a small piece of tubing, and at least one optical fiber. Oxygen pressure is monitored by measuring the lifetime of the oxygen sensitive oxyphor in solution within the chamber B. Published results from the inventors show that Oxyphor G2 and Oxyphor R2 can be directly correlated to the tissue O₂ levels in vivo. (Dunphy et al., Anal. Biochem. 310(2):191-198 (November 2002)). The oxyphor solution 3 is sealed in the chamber B and does not contact the surrounding medium, but the oxygen outside the chamber rapidly equilibrates with the Oxyphor solution within the chamber.

In the present invention, as well as in copending U.S. application Ser. No. 12/087,391 relating to the tissue oxygen sensor, the gas measurements are not made in a fluid or in blood within a blood vessel, although the tissue may itself be, and likely is, vascularized. Living tissues in the body of a patient are, indeed, vascularized, being richly supplied with capillaries. Many of the sensor, detection, and information recording components disclosed in U.S. Pat. No. 5,830,138 may be adapted for use in the present invention, and it is entirely incorporated by reference herein.

Nevertheless, the present system, and probe and methods of its operation are neither the same as the intra-vessel detection method of the '138 patent, nor does the present device require placement within the lumen of a vessel. The present invention is not intended for measuring arterial or venous blood gases. Thus, it is not limited as is the '138 invention, which requires insertion of the probe into a blood vessel. Nor is the present invention intended to operate in a primarily fluid environment, such as within blood in a blood vessel, although, of course, some fluid present within many tissues. But the comparison is in blood as a fluid, although is also contains cells, versus tissue which is not primarily fluid in vivo. To the contrary, the embodied tissue probe is specifically designed and intended to withstand the compression of surrounding non-fluid tissue, including dense muscle tissue, without damage, alteration or collapse of the probe A or the sensor chamber B contained therein.

In the case of the oxygen sensor, all devices for exciting the phosphor, and for reading the phosphorescence produced to determine the oxygen levels in the tissue may be activated and utilized, transdermally or by optical fiber connection, from outside of the patient. As above, as diodes become smaller, the excitation light source for the phosphors may also be self-contained and included within the probe end, rather than external to the patient. Such use of an internal excitation light source is further encompassed by the present invention.

The alternative embodiment of the tissue pH/CO₂ measuring system of the present invention comprises a sensor chamber B containing a solution of a pH/pCO₂-sensitive fluorophor analyte 3 within a biocompatible, gas-permeable membrane that quickly permits the analyte to assume the same pCO₂ concentration as the surrounding tissue. All devices for exciting the fluorophor, and for reading the fluorescence produced to determine the pH/pCO₂ levels in the tissue may be activated and utilized, transdermally or by optical fiber connection, from outside of the patient. Eventually, as diodes become smaller (<2 volts), the excitation light source may be self-contained and included within the probe end, rather than external to the patient. Such use of an internal excitation light source is further encompassed by the present invention.

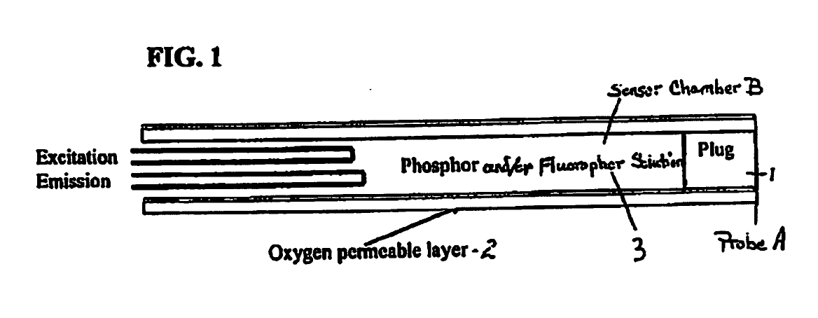

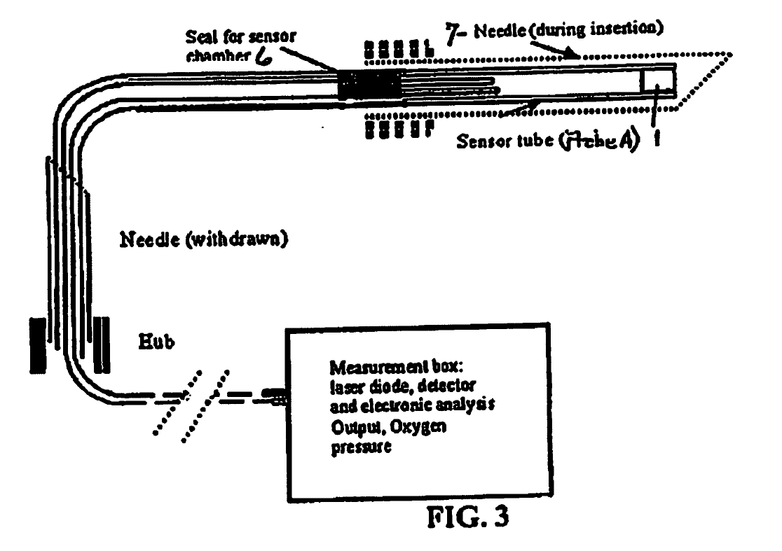

In a preferred mode of the current pH/CO₂ invention, as in the disclosed oxygen sensor, the probe containing the sensor chamber B and analyte(s) 3 is operably connected to optical fibers 4 and/or 5 for conducting the excitation light to the fluorophor or phosphor 3, and for conducting the fluorescence from the excited fluorophor to the pH/pCO₂ detector, or the oxygen-quenched phosphorescence from the excited phosphor to the detector. FIGS. 1 and 3. Although surgical placement may be used, the probe is at its simplest and most useful form for detecting either peripheral oxygen or pH/pCO₂, or both if the probe contains both sensor systems, in emergency situations when the probe follows into the tissue behind a retractable insertion needle 7. Moreover, this needle must be retractable to place the sensor chamber B in direct contact with the surrounding tissue. FIG. 3. The sensor chamber B is, therefore, necessarily smaller in diameter than the retractable needle 7. In addition, in most applications, the sensor can be quite short (˜1-2.5 cm in length), since the probe tip is only inserted a short distance into the tissue, typically to depths of not more than 2-3 inches (including 1, 1.1, 1.25, 1.5, 1.75, 2.0, 2.25, or 2.5 cm and variations thereof). For resuscitation applications, the sensor would only need to be inserted so that the outermost end is within 3 mm deep (ranging to 1, 2 or 3 mm) into the tissue. In other applications, the length can be much greater.

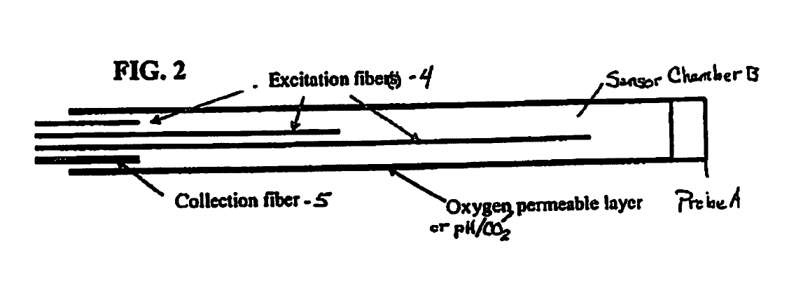

Tissue depth, however, should not be considered to be a limitation or requirement of the invention since alternative embodiments, for example comprising multiple sensor units, as will be described, may contain a plurality of sensors chambers distributed along the length of the fibers or at one or more points other than the distal tip of the fiber, allowing multiple measurements sequentially or simultaneously. Multiple optical fibers as shown in FIG. 2 may also be applied in the present invention to enhance and distribute the excitation light provided to the fluorophor and/or phosphor. In contrast to intravascular systems, e.g., the '138 patent, the entire length of that apparatus in the body must be covered with a catheter because it is exposed to the blood in the vessel. While the present invention does not require such a covering, it would, for example, be possible to coat the fiber optics with a sterile and sterilizable composition.

The “patient” of the present invention is any human or animal into which tissue oxygen measurement would be useful. The patient can be healthy or diseased, and be of any age or size, from neonates to adults. All will benefit from the advantages of the rapid and accurate measurement of tissue oxygen provided by the present invention.

Operable embodiments of the invention are described as follows. The invention, first described as an oxygen sensor (Heading 1 below), may be separately used, or combined with the alternative embodied pH/pCO₂ sensor (Heading 2 below). The remaining Headings apply to either and/or both embodiments, or to any embodiment disclosed herein.

### 1. System for Measuring Tissue Oxygen

An embodied system of the present invention comprises a biocompatible, gas-permeable layer-enclosed 2 sensor chamber B containing an aqueous phosphor analyte 3, which rapidly, in less than 15 seconds, equilibrates with the tissue oxygen of the surrounding tissue. When the phosphor is excited by a light source to phosphoresce, the level of resulting phosphorescence is modulated by the presence of oxygen in the surrounding tissue (oxygen-quenching is a well known characteristic of phosphorescence). Relying upon known physical properties of the selected phosphor, the oxygen-quenched phosphorescence lifetime of the analyte provides a highly accurate and direct measurement of the tissue oxygen level in the surrounding tissue.

Phosphorescent Compounds or Phosphors: Measurements in this embodiment are based upon the oxygen quenching of the phosphorescence of a phosphorescent compound having a known quenching constant and known lifespan at zero oxygen for a given temperature. Phosphorescence arises when a phosphor is excited to the triplet state by absorption of a photon of light and then returns to the ground state with emission of light (phosphorescence). The excited triplet state may also return to the ground state by colliding with, and transferring energy to, another molecule (quencher) in the environment. The rate of decay of the excited triplet state and phosphorescence lifetime depends on the concentration of the quenching molecules in the solution. In solutions with oxygen as the primary quencher (as it is in most biological sciences), the measured phosphorescence lifetime may be converted to oxygen pressure using the Stern-Volmer relationship. Repeated measurements can be used as a quantitative analysis of the time course of alterations in oxygen content in response to changed conditions. If the quenching constant and lifespan are unknown for a particular phosphor analyte, values can be determined by calibrating the quenching constant and lifetime at zero oxygen.

“Phosphors” or “phosphorescent compounds” of the present invention include any O₂⁻ sensitive compound (“Oxyphor”), which is soluble in the substrate being tested, and which upon excitation by a selected light source will produce a measurable phosphorescent light. In a homogeneous chamber, such as in the present invention, essentially all of the phosphor in the chamber should have the same lifetime, in contrast to heterogeneous oxygen distributions where information is found in the lifetime distribution. The phosphorescence lifetime of the excited phosphors suitable for the present invention is diminished or reduced (“quenched”) by O₂. The preferred selected phosphors contained in the sensors are hydrophilic or aqueously soluble, and generally biocompatible. Oxygen pressure is monitored by measuring the lifetime of the oxygen sensitive phosphor in solution within the chamber.

The phosphors employed in the present invention are preferably a material having: (1) a substantial sensitivity to oxygen, i.e. phosphorescence with high quantum yields at body temperature; and (2) a suitable phosphorescent lifetime, preferably on the order of from about 0.1 to about 1 msec to permit measurement. Although not intended to be limiting, suitable phosphorescent compounds include those described in U.S. Pat. No. 5,830,138 and co-pending U.S. Ser. No. 08/137,624, each of which is incorporated herein by reference, and as published in Vinogradov et al., J. Chem. Soc., Perkin Trans. 2:103-111 (1995). The phosphorescent compound is selected from the family of chemicals known as porphyrins, chlorins, bacteriochlorin, porphyrinogen, and their derivatives. Preferred porphyrins of the present invention include those hydrophilic compounds having the following formula:

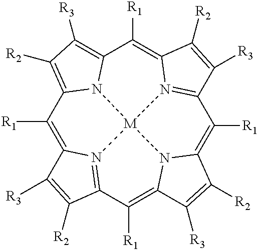

wherein R1 is a hydrogen atom or a substituted or unsubstituted aryl; R2 and R3 are independently hydrogen or are linked together to form substituted or unsubstituted aryl; and M is a metal. In certain preferred embodiments, M is a metal selected from the group consisting of Zn, Al, Sn, Y, La, Lu, Pd, Pt and derivatives thereof. Examples of such porphyrins, while not intended to be limiting, include, e.g., tetrabenzoporphyrin, tetranaphthoporphyrin, tetraanthraporphyrin, and derivatives thereof, e.g., meso-tetraphenylated derivatives; tetraphenyltetrabenzoporphyrins; tetraphenyltetranaphthoporphyrins; meso-tetra-(4-carboxylphenyl) porphyrins; meso-tetraphenyltetrabenzoporphyrins; meso-10 tetraphenyltetranaphthoporphyrins; and tetrabenzoporphyrins.

The preferred porphyrin structures are surrounded by a three-dimensional supramolecular structure known as a dendrimer. The dendrimer cage protects the porphyrin from quenching agents other than oxygen; and give it an appropriate quenching constant for oxygen (known in the art). It is known that one-, two-, and three-layer polyglutamate dendritic cages synthesized divergently around novel derivatized extended metalloporphyrin, oxygen-measuring, phosphor compounds provide phosphors which are highly water-soluble in a wide pH range and display a narrow distribution of phosphorescence lifetime in deoxygenated water solutions. More specifically, for use in the oxygen sensors of the present invention, dendritic derivatives of the aforementioned porphyrin phosphors are known, which are highly efficient and highly soluble phosphorescent compounds, encased in the dendrimer shell, and then coated with or surrounded by an inert globular structure, e.g., polyethylene glycol.

An example of such a compound is a derivatized metallotetrabenzoporphyrin compound, such as the Pd-complex of Pd-tetrabenzoporphyrin or Pd-meso-tetra-(4-carboxyphenyl) porphyrin. As disclosed in U.S. Pat. No. 4,947,850, incorporated herein by reference, substituent groups are known to impart desirable properties, such as solubility, to the preferred phosphorescent compounds. Formulation of preferred aqueous phosphorescent compounds of the present invention is provided in detail in the '138 patent, as incorporated herein. This design results in sensors that are water soluble and non-allergenic.

Oxyphors have been used to measure oxygen in the blood of many tissues in vivo, including the retina of the eye, brain, heart, muscle, liver and other tissues. The measurements can be made in animals that are either anesthetized or awake. In awake animals, however, the measurements are typically made through the skin, and therefore, limited to muscle, skin, or tumors, although mice are small enough for measurements of brain oxygenation. Because the Oxyphors have been designed to be impermeable to the walls of blood vessels, they can be injected into the blood for measuring in microcirculation or injected directly into the tissue for measuring in the interstitial space (see Wilson et al., J. Appl. Physiol. 101:1648-1656 (2006)). The injection of an Oxyphor directly into the blood stream has no effect on any of the measured physiological parameters of newborn piglets, rats or mice and adding it to cell growth media was found to have no effect on the growth of the cells, indicating no as yet detectable adverse biological effect.

In connection with the preferred substituted compounds of this embodiment, the inventors have found that substituent groups impart desirable properties to the compounds. For example, compounds which comprise substituent groups are characterized by solubility in polar solvents, including aprotic solvents, such as dimethylformamide (DMF), acetone and chloroform (CHCl₃), and protic solvents, such as water. The degree of substitution and the nature of the substituent groups may be tailored to obtain the desired degree of solubility and in the desired solvent or solvent mixture. The substituent groups are preferably substituted on the chromophobic portion of the compounds of the invention. The term “chromophobic portion” includes, for example, the atoms in the compound of formula I which are immediate to the porphyrin moiety, as well as the R1, R2 and R3 groups. Preferably, the substituent groups do not negatively affect or alter the absorbance and/or emission characteristics of the chromophores.

Two phosphors, one based on Pd-meso-tetra-(4-carboxyphenyl)porphyrin and the other on Pd-meso-tetra-(4-carboxyphenyl) tetrabenzo-porphyrin, are very well suited to in vivo oxygen measurements. Both of these phosphors are Generation 2 polyglutamic Pd-porphyrin-dendrimers, bearing 16 carboxylate groups on the outer layer. These phosphors are designated Oxyphor R2 and Oxyphor G2, respectively. See, Dunphy et al., Anal. Biochem. 310(2):191-198 (November 2002), incorporated herein in its entirety. Both phosphors are highly soluble in biological fluids, such as blood plasma and their ability to penetrate biological membranes is very low. The maxima in the absorption spectra are at 415 and 524 nm for Oxyphor R2 and 440 and 632 nm for Oxyphor G2, while emissions are near 700 and 800 nm, respectively. The calibration constants of the phosphors are essentially independent of pH in the physiological range (6.4 to 7.8). In vivo application has been demonstrated by using Oxyphor G2 to noninvasively determine the oxygen distribution in a subcutaneous tumor growing in rats.

More recently, Oxyphor G3, another Pd tetrabenzoporphyrin (PdTBP) modified with generation-3 polyarylglycine dendrons and coated with a layer of peripheral polyethylene glycol (PEG) residues has been developed and tested in the present invention. The dendrimer in G3 folds tightly around the PdTBP core in aqueous media and controls its exposure to oxygen. The phosphorescence quantum yield of G3 is about 2% and the lifetime T° is about 270 μsec. The Oxyphor G3 has absorption bands with maxima at 445 nm and 635 nm and the phosphorescence emission maximum is near 810 nm.

Preliminary measurements were performed using a solution containing Oxyphor dissolved in the medium within a small (250 micron inside diameter, 5 mm long) Teflon® AF chamber at the end of an optical fiber, which was connected to a prototype phosphorescence lifetime instrument. Tests for stability of the oxygen measurements were done by placing the chamber in a vial of air saturated with water and 2000 measurements were made at 10 second intervals. The data showed no significant loss of measurement capability over this period (5.5 hours). Sensor response time was determined by moving a sensor from a solution of one oxygen pressure (150 torr) to another (15 torr) and back while measuring at 4 second intervals. The full cycle time was less than 3 minutes for the sensor (50% response time of about 12 seconds).

As compared with prior art uses of oxygen-dependent quenching of phosphorescence, the present invention's use of sensor chambers eliminates the previously required injection of the phosphors into a patient's biological fluids. In the present invention, since the Oxyphor is in water solution at a concentration of 10 micromolar and the internal volume of the chamber is less than 1 microliter, very little Oxyphor (less than 10 picomoles) will be in the chamber. The Oxyphor remains in the chamber while in the tissue, and is entirely removed with the optical fiber and chamber when the measurements are no longer needed. However, even at such small amounts, none of the Oxyphor ever mixes with the tissue or body fluids of the patient. To the contrary, the analyte is now contained within the sensor chamber B, and is therefore easily removed from the patient's body after use.

When the phosphor-containing sensor solution is exposed to a modulated light capable of exciting the phosphor to emit phosphorescent light, measurement and calibration of both the phosphorescence intensity and delay time between the excitation light intensity and the phosphorescence emission (signal) is effected. Therefore, accurate determination of the frequency dependence of the signal amplitude and phase is used to calculate the oxygen pressure histogram of the sample using algorithms. The measured oxygen pressure histogram can then be used to accurately calculate the oxygen concentration gradient throughout the sample.

Phosphorescence quenching has been thoroughly verified as a method of measuring the oxygen dependence of cellular respiration (see, for example, Vanderkooi and Wilson, “A New Method for Measuring Oxygen Concentration of Biological Systems, in Oxygen Transport to Tissue VIII, Longmuir, ed., Plenum (August 1986); Vanderkooi et al., J. Biol. Chem. 262(12):5476-5482 (April 1987); Wilson et al., J. Biol. Chem., 263:2712-2718 (1988); Robiolio et al., Am. J. Physiol. 256 (6 Pt 1):C1207-1213 (June 1989); Wilson et al., Adv. Exp. Med. Biol. 316:341-346 (1992); and Pawlowski et al., Adv. Exp. Med. Biol. 316:179-185 (1992). Detailed data on the calibration techniques and oxygen measurement capabilities of a widely used phosphor is provided in Lo et al., Anal. Biochem. 236:153-160 (1996). At constant temperature, phosphorescence lifetime is independent of the other parameters and composition of the sample.

It is important in the present embodiments to use a compound of known quenching constant and known lifetime at zero oxygen for a given temperature. Thus, once the compound and temperature are determined, calibration need only be made on a single occasion, after which the value can be used for all subsequent measurements involving that compound

Since temperature is an element of the calculation, temperature sensors used may be included in this system using micro-versions of temperature sensitive resistors, thermocouples or thermisters embedded in a thin coat of biocompatible plastic. These are placed very near the oxygen and/or pH/CO₂ sensor, with the wires preferably embedded in the plastic coat of the optical fiber. In other embodiments the temperature sensor is separated from the oxygen and/or pH/CO₂ sensor(s) and enclosed in a small tube, which could be microbore tubing made of Teflon or other appropriate plastic tubing. This tubing is either placed separately in the patient, or bound to the optical fiber and placed together with the optical probe.

Calibration of the phosphors is absolute, and once phosphors have been calibrated in one laboratory the same constants can be used by anyone else as long as the measurement is done under the same conditions. The measurements are rapid and highly reproducible. Less than 2 seconds are required for each measurement, and current instruments have a measurement-to-measurement variability of less than 1 part in 1000. Due to the absolute calibration, equally low variability is attained among different tissue samples having the same oxygen pressure.

Increasing the concentration of Oxyphor increases the absorption of excitation light in the chamber. With an absorption coefficient of approximately 50 cm⁻¹ mM⁻¹, a solution with a concentration of Oxyphor of 10 μM, the solution will absorb 70% of the excitation light/cm. Measuring observed phosphorescence signal as a function of the Oxyphor concentration over a range of 1 to 100 μM, optimal is expected near 20 μM.

The measured phosphorescence lifetime values are then used to calculate oxygen pressure from the Stern-Volmer relationship T°/T=1+k_{Q}T°[pO₂] (Equation 10). In this relationship, T° is the lifetime in the absence of oxygen, T is the lifetime at a given value of oxygen pressure (pO₂), and k_{Q} is a constant describing the frequency of quenching collisions between the phosphor molecules in the triplet state and molecular oxygen. k_{Q} is a function of the diffusion constants for phosphor and oxygen, temperature and phosphor environment, determined by calibration of the phosphorescence lifetime at the temperature of the measurement.

Thus, the system allows for the in vivo measurement of the probability of an excited triplet state phosphor colliding with an oxygen molecule from its surrounding environment. An increased number of oxygen molecules in the surrounding medium correspondingly increase the probability of collision. Because concentration is a measurement of the quantity of a desired object per unit volume of a particular medium, the system therefore allows for a measurement of in vivo oxygen concentration of peripheral tissue. However, as is well known to those skilled in the art, at equilibrium, oxygen concentration is proportional to oxygen pressure in the gas phase. Therefore, because atmospheric oxygen pressure is known, pressure calibrations may be made and then concentration calculated from tables of oxygen solubility at a given pressure and temperature. In this way, a measurement of oxygen pressure is easily converted to a measurement of oxygen concentration and vice versa. As such, all references to “oxygen” and “oxygen pressure” measurements contained herein are not limited to oxygen pressure or oxygen concentration alone, but are intended to encompass both, as one skilled in the art may easily ascertain both measurements, given the data provided by the system.

The present system, therefore, comprises all of the elements necessary for measuring tissue oxygen: the sensor probe A including the gas-permeable layer 2 and the analyte 3, a light source, a photodetector, and further in the case of the system using optical fibers 4 and 5, one or more optical fibers operably connected to deliver excitation light from the light, and for collecting and delivering phosphorescence to the photodetector from which oxygen pressure can be calculated based on the oxygen quenching of the analyte activity.

### 2. A System for Measuring Tissue pH/CO₂

In the alternative embodiment of the invention tissue pH/CO₂ is directly measured in the tissue by the response of an aqueously soluble, highly fluorescent pH indicator, referred to as a “fluorophore” or “fluorescent compound” 3, in the sensor chamber B. Aqueous fluorescent compounds of any type known in the art may be used having a pK between 5 and 7.5. The fluorophor fluoresces at the same wavelength, but absorbs at different wavelengths in the protonated and unprotonated forms, involving the hydrogen ion concentration. Two different wavelengths are used so the ratio of the protonated and unprotonated forms can be calculated directly from the ratio of the fluorescence at the two different excitation wavelengths. A ratio of the intensities of the two forms is calculated as a ratio of the excitation of the protonated form, as compared with the unprotonated form. This ratio, plus the pK, is all that is needed to calculate pH, eliminating the need for calibration. By comparison, the oxygen measurements using the sensor described herein utilizes only one wavelength for excitation and emission in the oxygen measurements, and is calculated differently.

For example, for the pH/CO₂ sensor, a mechanical adaptation is constructed to optimize assembly of the LED, interference filter, and optical filter fibrous light guide, below, which are connected to a fiber optic switch to send the beam for excitation of the fluorophor, or to a photodiode detector to measure relative intensities of analyte excitation at multiple wavelengths. This allows the ratio of the fluorescence at the two different excitation wavelengths to be used as a measure of pH, which provides a measure of the CO₂ concentration in the tissue in accordance with the previously identified Equations 1-9.

As long as the relative intensities of excitation light of the two different wavelengths is known, the measured pH values are independent of the concentration of fluorophor, the intensity of the excitation light, and the efficiency of collection of the emitted fluorescence. The measured excitation energies are used to correct the fluorescence intensity ratio for the equal energy of the two wavelengths. After switching, excitation light is passed into a 50:50 coupler with a common end terminated with a connector designed for rapid and reproducible connection of a fiber optic means, for example, connected to the sensor chamber B. This is only for the pH sensor since there is only one wavelength for excitation and emission in the oxygen measurements.

This system may also be combined with the measurement of pO₂. In addition to the fluorophores or fluorescent compounds, phosphors or phosphorescent compounds, as described herein, may also be added in a separate sensor analyte or as a combined sensor analyte in solution to measure both pO₂ and pH/CO₂ or K⁺ levels in the tissue upon simultaneous or sequential excitation of either, or both, the phosphor and/or the fluorophor.

### 3. Optical Fibers

In certain embodiments of the invention, one or more optic fibers 4 are used to provide light transmission through flexible transmission fibers to direct the light to the distal end of the sensor probe. In that case, the wave-guide is a single optical fiber or several single fibers, or a bundle of light conducting fibers, or any combination thereof (collectively referred to herein simply as an “optical fiber”). The amount of light that will enter the fiber is a function of several factors: the intensity of the light source (e.g., LED or LD), the area of the light emitting surface, the acceptance angle of the fiber, and the losses due to reflections and scattering. As the term is typically used, each optical fiber comprises a light carrying core and cladding which traps light in the core. Usually each fiber is a two-layered, glass or plastic structure, with a higher refractive index interior covered by a lower refractive index layer. One of ordinary skill in the field of fiber optics would be familiar with, and could readily select from, the range of construction types, from continuous gradient to steps in refractive index. If cladded it would be specifically adapted for the present invention, as in a permeable, but reflective, plastic film layer.

The optical fibers for conducting the excitation light to the fluorophor and/or phosphor and, as described below, for conducting the fluorescence and/or phosphorescence from the phosphor to the detector, are connected to the phosphorescence lifetime measuring instrument through, e.g., a dual channel quick connect port, making the light guide element easily connected and disconnected from the fluorescence or phosphorescence lifetime measuring instrument. See FIG. 3. The term “light guide,” used interchangeably with wave-guide or optical-guide, and spelling variations thereof, is used herein to refer to a light conductive element that provides light of the necessary wavelength(s) to be used in connection with the sensors and the system of the present invention. The waveguide allows transmission of light into the patient's body to excite the analyte so that the emitted light can be detected externally, from outside the body.

As exemplified, the refractive index (r.i.) of the selected analyte solution in the sensor chamber B is chosen to be as near, or if possible, substantially identical, to that of the optical fiber, to permit it to become in effect an extension of the optical fiber means for increased efficiency of emitted light transfer through the optical fiber to the detector. Again without intending to limit the present invention to any particular theory, it is known that optical fibers conduct light because the internal refractive index is much higher than that of the environment outside the fiber. For example, the refractive index of air is approximately 1.0, while that of typical optical fiber is about 1.5. This difference means that the fiber collection angle is about 60°. In other words, light approaching the fiber wall from the inside at angles up to 30° (½ the collection angle) is reflected back into the fiber and continues to travel along the fiber. This is also the case for a thin tube filled with a high refractive index solution, and efficient light guides constructed in this manner are known. See, for example, Oriel Corp., Stratford, Conn. There are many liquids known to possess refractive indices (r.i) high enough for forming light guides, such as, for example, possessing a refractive index higher then about 1.4, e.g., 40%-80% sucrose in water (r.i.=1.40 to 1.49), glycerol (r.i.=1.47) or mineral oil (paraffin oil) (r.i. 1.47) as compared with water (r.i.=1.33) using communication grade acrylic fiber optics with a core refractive index of 1.495 and a collection (‘acceptance’) angle of 60°.

Suitable plastic for the optical fibers include, e.g., but without limitation, polymethylmethacrylate, or one having a silica light core, which is of a size suitable for entry into a tissue area to be tested. The fiber core diameter for the exemplified laser light is preferably less than 200 microns. Fibers for collecting the phosphorescence are about the same size, ranging up to 400 microns.

In an alternative embodiment, the optical fiber(s) are encased with a sleeve of a biocompatible, but suitably inert material, such as a plastic for a portion thereof before and after leaving the sensor chamber B. To provide greater rigidity and durability, the gas-permeable sleeve 2 preferably has a portion which overlaps an end portion of a probe means of a corresponding length, and in which a portion of overlap can be, for example, fusion sealed to form a probe A containing at least the gas-permeable layer 2 enclosed sensor chamber B. For protection and durability, the end of the probe adjacent to the needle 7 can be reinforced with a plug 2 or other protective covering. See FIGS. 1-3.

Wireless: In an embodiment of the invention, the device is as described, but instead of connecting to an optical fiber in the probe for transmitting the excitation light to the sensor analyte, those fibers and connections are removed, thereby creating a wireless system. In this alternative embodiment, the sensor molecules are selected to have absorption and emission bands in the near infrared region of the spectrum (absorption between 600 nm and 850 nm and emission between 630 nm and 1300 nm). The selected light source, such as an LED, is then placed on the patient's skin in closest proximity to the tube inserted or implanted in the patient's tissue up to 1 or 2 cm deep in the tissue, wherein the tube contains the gas-permeable layer 2 covered, analyte-filled sensor chamber B (the probe element A of the system). In other words, the optical fibers for the excitation light are replaced by the near infrared emitting LED that transmits the light transdermally through the patient's skin. This operation is more readily adapted to oxygen measurements in which using phosphorescence lifetimes for oxygen measurements, using phosphorescence allows elimination of the light scattering and tissue fluorescence contributions. However, the same principle could be adapted for the pH/CO2 measurement, although it would be more difficult since fluorescence intensities are used for pH/CO2 measurement, instead of phosphorescence lifetimes.

Similarly, to permit the removal of all outside connections to the probe, the remaining collection optical fibers for transmitting the analyte-emitted light to the detector are also removed from the present system. Instead, the detection device (CCD or equivalent) are place on the surface of the patient's skin in closest proximity to the probe element of the system. This embodiment relies on the ability of near infrared light to penetrate substantial thicknesses of tissue due to the low level of absorbing pigments at these wavelengths.

In practice, the light from the LED penetrates the patient's skin and surface tissue, striking the analyte filled tube or probe and exciting the analyte to produce measurable levels of oxygen quenched phosphorescence and/or pH-indicating fluorescence. In such an embodiment, the excitation and emission light can independently pass through thicknesses of one or more centimeters of skin or tissue if the excitation light is delivered wirelessly and transdermally, from outside of the patient to a probe positioned within the tissue. Then in a reverse process, the emitted phosphorescent and/or fluorescent light is returned to through the patient's surface tissue and skin to the detector device. An adequate signal is transmitted by measuring for low phosphorescence levels, by using a sufficiently high concentration of phosphor in the sensor chamber B, by using a bright LED to produce the excitation light, and by keeping the sensor tube within less than 1 cm, or not more than 2 cm, of the skin surface or outer surface of an organ, muscle, or whatever tissue is being analyzed. Such a wireless, surface system for measuring tissue oxygen would be particularly effective for use for resuscitation and emergency care. Of course, such a wireless system may not necessarily ideal for all situations, whereas the full system with fibers offers broader application.

### 4. Sensor Chamber

A small sensor chamber B contains a solution of the pH/pCO₂ analyte and/or the tissue oxygen sensitive analyte, typically within a small diameter tube of a biocompatible, gas-permeable material. The tubing has a outer diameter preferably about 400 microns, although diameters from 50 to 1,000 may be used, preferably ranging from ranging 200-500 micron, including diameters of 100, 200, 300, 400, 500, 600, 700, 800, or 900 micron. The inside diameter of the tubing or fiber averages 200 micron (ranging 50-300 micron, including 100, 150, 250 micron ranges, and other values in-between). Typical length is 6 mm (ranging 2-7 mm, including 2, 2.5, 3, 3.5, 4, 4.5, 5, 4.5, 5, 5.5, 6, 6.5 or 7 mm and the like) in length, and has an internal volume of ≦1 microliter (including 0.1, 0.2, 0.3, 0.4, 0.5, 0.6, 0.7, 0.8 0.9, 1.0 microliter). Further, a piece of tubing this size is able to pass through an 18 to 22 gauge needle 7 for placement in the patient's peripheral muscle tissue. Preferably a 20 gauge needle is used, as compared to one that is 18 or 19 gauge. As the sensors get smaller the needles also get smaller. Recent chamber tubing is smaller (420 micron outside diameter) than what was used for the first prototypes (500 micron outside diameter), meaning that the needle gauge can decrease from 20 or 21 gauge to, so far, as small as 22 gauge (larger gauge means smaller needle), and will continue to decrease directly with the decreasing outer diameter of the sensor. Thus, the proposed sensor is of a smaller diameter than a human hair, making tissue insertion possible, and relatively painless.

The pH/pCO₂ and oxygen sensitive analytes may be combined if they do not interact or adversely affect the results of either analysis. In the alternative, two sensor chambers B are positioned in the resulting system, each containing a different analyte to provide both pH/pCO₂ and tissue oxygen measurements, one in each chamber, without any interference or interaction. A gas-permeable layer 2, such as a plastic, is selected that neither absorbs the excitation light, nor the fluorescent and/or phosphorescent light from the sensor. Thus, the selected plastic requires a low solubility for pH/pCO₂ or oxygen and a low diffusibility of pH/pCO₂ or oxygen. The material(s) of construction of the gas-permeable layer 2 is not critical to practice of the invention, so long as it meets the necessary requirements, and can utilize any acceptable known material, including, but not limited to, such plastic layers as silastic, Teflon® polyethylene and polypropylene, so long as the layer does not inhibit gas permeability to the sensor analyte and meets the requirements for the transmitted light. Teflon is used in the exemplified embodiment as described in greater detail below.

As indicated, the analyte solution containing the sensor is preferably aqueous. The aqueous sensor is, therefore, further described herein in terms of the phosphorescent analyte for oxygen detection, although as indicated below, a fluorescent sensor follows the same principles and may also be added to the sensor chamber B or combined in a separate sensor chamber B. Although fluorescent and/or phosphorescence is emitted uniformly in all directions, in the present invention, the fluorescent and/or phosphorescent solution preferably has a higher refractive index than that of the gas permeable layer (plastic in the wall of the tube), such that the solution acts as a light pipe or guide. Fluorescent or phosphorescent light, which is emitted at angles less than the collection angle of the light pipe (numerical aperture), is thus refracted back into the solution and along the tube. In other words, because the refractive index of the solution (having a high refractive index core) is higher than that of the wall of the plastic tubing (having a low refractive index core), transmitted light hits the wall at less than the refraction angle. Thus, light is refracted back into the solution.

When optical fibers are used, excitation light (for the purposes of either causing fluorescence and/or phosphorescence) is delivered to the sensor solution by the excitation fiber, and is thus channeled down the fiber core, rather than exiting from the sides. This confines the light to the solution (causing a light pipe effect within the gas-permeable tube layer 2 surrounding the sensor and fiber(s)). As a result, the efficiency of exciting the fluorophor and/or phosphor is greatly enhanced because less light is lost through the wall of the sensor, or of the optical fibers. Moreover, the efficiency of collecting the fluorescence and/or phosphorescence emitted from the excited fluorophor and/or phosphor 3 in the sensor chamber B is greatly enhanced.

Unless wireless, the excitation delivery and collecting optical fibers, respectively, are operably connected and sealed 6 to the proximal end of the sensor tube containing the analyte solution (the probe for either pO₂ and/or pH/pCO₂). In the method of operation of an exemplified embodiment, the excitation light travels along the tube to the fluorophor and/or phosphor in solution in the sensor chamber B within the probe A positioned in the tissue, and then following excitation of the fluorophor and/or phosphor, the fluorescence and/or the oxygen-quenched, emitted phosphorescence light is transmitted back along the tube, where it is collected by the collection optical fiber and delivered to the detector.

There are several embodiments of the tube that are useful for specific applications. In one embodiment, for example, several fibers are provided for excitation of different lengths of time or wavelengths. The excitation light may be applied through each of the plurality of individual fibers in sequence. In another embodiment, depending on the concentration of the analyte (absorption of the excitation wavelength), a single collection fiber is used. In the alternative, however, a collection fiber may be provided for each corresponding excitation fiber.

In yet another embodiment, the excitation light may be further confined to a short region of the tube near the end of the fiber for absorption by the fluorophor and/or phosphor, whereas emitted longer wavelength phosphorescence could travel longer distances through the sensor solution. Thus, the fluorescence and/or phosphorescence lifetime (equating to oxygen pressure) is measured for each excitation site along the tube. For longer distances the number of collection fibers are increased as needed, although optimally there is one collection fiber (or fiber site) per excitation fiber (or fiber site).

An important consideration when constructing very small chambers B for either pCO₂ and/or oxygen measurements is obtaining enough of a signal to provide accurate measurement, and using materials that allow rapid equilibration of the oxygen and carbon dioxide outside the chamber with the solution within the chamber. An exemplary material for this purpose is Dupont's Teflon®, in particular Teflon® AF. Teflon® AF is also known to function as a sensitive optical fiber. In particular Teflon® AF is highly permeable to oxygen, as well as to pH/pCO₂. The use of the low refractive index Teflon® AF for the sensor tubing makes the refractive index of water sufficient to produce a good light guide effect.

In addition to chemical inertness and other characteristics commonly found in Teflon®, this material has a very low refractive index (lower than water) and tubes formed from this material have a very smooth internal surface. As such, when the tube is filled with a liquid having a higher refractive index than Teflon® (such as water), the liquid becomes the core of a light guide. Light entering at one end nearly parallel to the tubing will be internally refracted and travel along the tubing in the liquid core instead of exiting through the wall of the tube, just as light travels through the core of more conventional optical fibers. Because of this, not only is the efficiency of excitation of pH and/or oxygen indicator in the liquid contained within the tube increased, but the collection of emitted fluorescence or phosphorescence is increased as well.

Because of the side chains in polymeric Teflon® AF, this material has a much higher permeability to gases, such as oxygen and carbon dioxide, than the regular form of Teflon®. This permeability is comparable to that of tubing formed from silastic, but the Teflon® AF tubing has a much greater strength and structural stability. It is believed that the ability of the Teflon® tubing to resist bend induced “collapse” decreases with greater length. Although, in general, longer fiber optic light guides provide better signals, initial measurements suggest that there is no optical advantage to Teflon® AF tubing lengths greater than about 6 mm. Further, experience so far indicates that Teflon® AF tubing with an exemplary inside diameter of 140 microns and an outside diameter of 320 microns, gives sufficient signal. The strength of the optical fiber and the chamber decreases when smaller sizes are used. Additionally, with decreasing size comes an increase in the technical challenges of construction and measurement.

As such, the above-mentioned 6 mm length of tubing is exemplified, as this length provides good signal, yet the tubing remains quite strong and is not easily bent. There is no intent, however, to in any way limit the invention to a 6 mm length, as other suitable lengths may be apparent to those skilled in the art with the benefit of this disclosure. Such other lengths and smaller sizes may be advantageous for specific clinical uses such as in pediatrics, particularly newborn infants.

Although Teflon® AF tubing is embodied in the exemplified system, the invention is in no way so limited to chambers B made of this material. It is anticipated that such chambers will be created from materials with gas permeability properties similar to those of Teflon® AF, as would be apparent to those skilled in the art with the benefit of this disclosure.

The chamber is sealed (plugged) with two pieces of optical fiber, one piece inserted into each end of the tube. See, FIG. 1, elements 1 and 6. High yield of the plugged chambers are obtained, for example, by sealing empty Teflon® tubing onto the end of an optical fiber and then placing several of these in a vacuum chamber with a solution of the Oxyphor and/or fluorophor. When making the chambers it is important to avoid getting air bubbles inside the chamber. In one embodiment, the optical fiber has a 250 micron outside diameter while the Teflon® AF tubing has a 250 micron inside diameter. This configuration allows for a water tight seal 6 with good stability, although the strength can be increased by using Teflon® compatible glue on the plastic fiber, a heat seal, or other commercially accessible and medically approved method for sealing. Silica fibers are substantially strengthened by coating them with medical grade high strength plastic polymer. Optimization of the sensors is achieved through critical comparisons of the strength, resistance to bend breakage, and light transmission properties of plastic and silica optical fibers in conjunction with varied concentrations of pH. Initial testing has demonstrated that 250 micron plastic fibers worked well for transmitting a signal from a coupled laser diode.

However, other forms of Teflon®, as well as other plastics, may also be used. The only requisite criteria are that the oxygen permeability of the tube must be high enough that the sensor solution can rapidly respond to alterations in the oxygen pressure in the tissue (“tissue oxygen pressure”) and/or to the tissue pH/CO₂, and that the response is sufficiently rapid for the particular application. In general, the following characteristics each enhance the performance of the sensor: thinner wall; smaller tube; and higher oxygen permeability of the wall material. The effect of each enhancement is cumulative if combined. Should the response times in tissue prove to be longer than desired, smaller diameter and/or thinner walled tubing is provided. Response time may begin simultaneously with insertion of the probe into the tissue, or may begin at any time chosen. Present response times for a tube having 500 micron outside diameter is about one minute, but it is expected that as size is reduced to about 400 micron outside diameter, allowing insertion through 22 gauge needles, that response times will drop to 30 seconds or less, preferably less than 20 seconds (to place and provide accurate measurements). Both the amount of oxygen that needs to diffuse into/out of the chamber and the diffusion distance decrease and as the diameter and wall thickness decrease.

For the pCO₂ measurement system, the amount of CO₂ that has to diffuse through the tubing is significant. The amount of CO₂ that needs to diffuse into/out of the chamber is a function of the concentration of bicarbonate buffer inside the tube. The lower the bicarbonate concentration the more rapidly the sensor will respond. As was the case for the oxygen sensor, smaller tubing and thinner walls will also result in faster equilibration times and more rapid response to changes in external CO₂ pressure. It is also possible to include PEGylated carbonic anhydrase in the sensor medium to catalyze equilibration of the CO₂ with bicarbonate. Carbonic anhydrase is available in very active and stable forms, meaning that adding it to the solution in the chamber is a feasible if equilibration significantly slows the sensor response time.

The fiber optic sensor chamber B can be made entirely of medically approved materials. It is easily sterilized, and may be stored for months at room temperature without loss of function. Methods used for filling the fiber optic sensor chamber B are described with regard to the above-mentioned solutions. See, e.g., Oxygenase, LLC Proposal for Small Business Technology Transfer (STTR) Program, Topic A09A-T027; Proposal A09A-027-0192.

Testing of the sensors: After evacuation to <0.005 atmosphere the Teflon® tubing at the end of the fibers was placed in the Oxyphor solution and the vacuum released. The Teflon® tubing fills completely and the filled units are then removed from the chamber and the open end sealed as described above. The thus-filled chambers have been tested for stability of the oxygen measurements by placing the vial of air saturated water. 2000 measurements were made at 10 second intervals. There was found to be no significant loss of measurement capability in 2000 measurements (5 hours). Sensors are stable in situ for at least 1 day of continuous measurements (7000 measurements) or for 2 days at physiological oxygen pressures, the longest time these sensors are likely to be left in place.

### 5. Extendable/Retractable Insertion Needle

Unless the probe is surgically implanted, the device further comprises a resilient, extendable/retractable insertion needle 7 of a size and shape suitable for use in tissue to facilitate insertion of the sensor probe through the skin and into the patient's tissue. See FIG. 3. For example, the needle 7 is designed to permit insertion of the sensor probe through the skin into the tissue beneath it, including muscle or other dense tissue, or through the abdominal wall into an underlying organ. Such a needle 7 has an inner and outer surface and in one embodiment a round cross-section, but the needle is not limited to a round cross-section, and may be oval, square or otherwise, depending on the shape of the probe. The size of the needle may match the size of the probe (preferably 20, 21 or 22 gauge) and guides the probe's inward motion into and through the tissue, including skin and internal organs.

In any embodiment of the invention, known needle retraction mechanisms may be used, whereby after entry of the probe, the needle guide is retracted from the projecting position to a position posterior relative to the sensor probe tip. The insertion needle 7 for inserting the probe into tissue would be similar to retractable needles already known in the art, e.g., as used for inserting intravenous catheters. A suitable needle retraction mechanism for use in the present invention may be similar to one of those disclosed in U.S. Pat. No. 5,782,804, which is incorporated herein by reference, although the cited patent is different in that it refers to needles for liquid delivery or delivery to a vessel. Retraction may be internal or external, but is preferably external to the probe, and may include one or more elastomeric or resilient ring members to operably seal the sensor probe and the needle member. See, e.g., FIG. 3.

The needle 7 has a central hole larger than the external diameter of the sensor tube containing the oxygen sensitive phosphor solution. The gas-permeable layer 2 covered sensor chamber B sits within the needle 7 or extends proximally from the needle during insertion. Such needles typically have an inner diameter within a range of approximately 0.002 inch to 0.010 inch and an outer diameter within the range of approximately 0.004 inch to 0.012 inch. The purpose of the needle 7 is simply to offer protection for the sensor chamber B when it penetrates the skin and/or enters the tissue. It adds strength and sharpness, and then is pulled back out of the way, so that it is not directly involved in the system for measuring tissue oxygen concentration. It is used only for transport purposes for the sensor chamber B. Practitioners in the medical field are familiar with many similar devices, as used for the leading end of catheters, etc. In a specific embodiment, the needle catheter may include a sensing capability to determine penetration depth of the needle, as well as dial-in needle extension.

### 6. Excitation Light

As indicated above, the system also comprises an excitation light source, preferably a modulated light source, is employed for excitation of the soluble fluorophor and/or phosphor compound(s) in the sample to a state of fluorescence and/or phosphorescence for measurement. The light source means can be provided by any of several different sources, including a flash lamp, a pulsed light emitting diode, or a pulsed laser. The designs of the light source and/or detector, in accordance with this invention, are not critical to the practice of this invention and may take any suitable form employing any conventional and non-conventional components. A beam of excitation light is passed through the sensor solution (analyte) from any direction, but as embodied, through the light tube, so long as the beam passes completely throughout the sensor. The emitted fluorescence and/or phosphorescence is then collected from any point, but as embodied, through the light tube.

A laser diode, which in certain embodiments is coupled into an optical fiber 4 (6 to 250 micron core diameter), is preferably used for excitation of phosphorescence in a phosphor sensor of the probe. A light sensitive detector (photomultiplier, avalanche photodiode or silicon photodiode) is used to measure the emitted phosphorescence. The detector is preferably covered with an optical filter to exclude light of all wavelengths other than that of the emitted phosphorescence. In particularly, the wavelength(s) of the excitation light is excluded from those wavelengths that are detected. The laser diode applies light, either in short pulses (time domain measurement of lifetime) or modulated at differing frequencies (frequency domain measurement of lifetime). In each case, the detected phosphorescence signal, fitted to a single exponential or the phase shift relative to the excitation light, is determined and used to calculate the oxygen concentration in the tissue.

In one exemplified embodiment, the excitation light is applied as a flash of monochromatic light (a width at half-maximal intensity of less than 5 microseconds for flashlamps), i.e., undulated sinusoidally, from 20 to 50,000 Hz, preferably from 50 to 35,000 Hz, most preferably from 100 to 20,000 Hz, filtered to provide the desired wavelength, i.e., between 400 and 700 nanometers. The preferred measurements detect only those emissions that are at a longer wavelength and modulated at the same frequency. In another exemplified embodiment, the light source is a light-emitting diode (LED), such as a laser diode. LEDs provide monochromatic light with a relatively broad bandwidth. Such light is passed through an interference filter, thus blocking the long wavelength “tail” in the emission of the LED, which might otherwise interfere with fluorescent measurements of the present invention. The separation of excitation and emissions wavelengths of fluorophores and/or oxygen-quenchable phosphors is generally sufficient to not require such a filter. Ideally, all light emitted from an LED or laser diode (LD) would be at the peak wavelength. But in practice, light is emitted in a range of wavelengths centered at the peak wavelength. This range is referred to as the “spectral width” of the source.

Solid state light sources can be readily modulated at the desired frequency and are monochromatic, i.e., light emission occurs primarily in either a broad band up to about 60 nm bandwidth at half-height for LEDs, or at a narrow band of 1 nm or less for laser diodes. As a result, minimal optical filtering is required for optimal application of such light to the measurement of phosphorescence levels. Modulation of the light can be achieved either by direct modulation of the light source or by passing the light through a modulation device, such as a flasher or a rotating wheel with slots through which the light may pass.

The excitation light source is in one embodiment, a fiber coupled laser diode (LD), although continuing advances in technology mean fiber coupled light-emitting diodes (LEDs) of sufficient intensity may soon become available. In another embodiment, the laser diode excitation light source is modulated at two frequencies at the same time (the LD driving waveform will be the sum of two frequencies of equal amplitude). Use of a low intensity and durable light source can be of significant advantage with respect to long term stability and reliability of the instruments. Interference from ambient light is greatly decreased by this method, since only signals with the same modulation frequency as the excitation light are amplified, which largely eliminates interference by other ambient light sources.

One of the frequencies selected to give a phase shift for measuring oxygen concentration uses a corrected phosphorescence signal of 28°, usually between 100 and 3,000 Hz, while the other is a frequency near 20 kHz. The exemplified 20 kHz signal is used as a measure of the “in phase” signal that can arise through leakage of the excitation light or fluorescence and/or phosphorescence. Because of its long lifetime (30 to 255 μsec), the phosphorescence signal at the low frequency excitation is nearly 100% modulated, whereas at 20 kHz it has a very large phase shift and minimal modulation (becomes nearly a constant value). At the higher frequency the measured emission signal modulation is almost entirely due to scattered excitation light and contaminating fluorescence and this has a phase shift near zero degrees. Simple mathematical algorithms can be used to accurately calculate the total “in phase” signal due to fluorescence and light scattering and using this to correct the phosphorescence lifetime determination.

In contrast, fluorescence and excitation have lifetimes of <100 nsec, and measurement is limited by the rise time of the amplifier (<3 μsec). Therefore, the “in phase” signal has the same amplitude and phase shift)(0°) at both frequencies.

Phosphorescence lifetimes calculated after correction for the “in phase” signal are not affected by the presence of such signals or by the presence of other chromophores, such as hemoglobin, as long as their absorption does not change in concert with the phosphorescence decay (<1 msec and repetitively), and extraordinarily unlikely behavior. This makes the method particularly effective for measuring oxygen in real-time in tissue in vivo. Phosphorescence lifetime is independent of phosphor concentration because there is no significant self quenching at the concentrations used, as long as the “in phase” component is less than about 50% of the total signal. Typically, the “in phase” signal is typically only about 5% as large as the phosphorescence signal.

The phosphorescence lifetimes are calculated assuming a single exponential decay (a single phosphorescence lifetime) and converted to the oxygen pressure using the Stern-Volmer equation. As shown, using the Stern-Volmer equation, intensities (amplitudes) of phosphorescent signals increase with decreasing oxygen pressures. Thus, the accuracy with which the phosphorescence can be measured increases as the oxygen pressure decreases, a characteristic that is particularly useful for in vivo measurements where pathology is associated with tissue hypoxia. See, STTR Proposal A-09A-027-0192, supra.

In one embodiment, the excitation light source of the system is small enough to be powered by a battery, offering advantageous portability to the use of the device. The power requirement is expected to be consistent with operation for up to 24 hours using a 12 V rechargeable battery. The laser optical power is less than 5 mW through the fiber, the optical power typically used for laser pointers.

### 7. Combiner/Splitter

The respective tissue oxygen and/or pH/pCO₂ systems also comprise a combiner for coupling the laser diode and photodiode to the optical fiber leading to the fiber optic sensor chamber B. As with any system designed for portability and for use by emergency responders, among others, maintaining the efficiency of the system, while at the same time reducing the overall size of the system, remains an important goal. Towards this end, the system works most effectively if one optical fiber can be used to convey not only the excitation light, but the returned fluorescence and/or phosphorescence, as well. By using only one optical fiber to connect the instrumentation box to the fiber optic sensor chamber, overall efficiency of excitation and collection of fluorescence is maximized.

There are two preferred ways to design the combiner. In one embodiment, the combiner is constructed of fibers that are much smaller in size than 250 microns, such as 200, 150, 100, 50 or 25 microns, preferably about 50 microns. At the common end (that which connects to the optical fiber leading to the fiber optic sensor chamber) the fibers are mixed and form a bundle with a maximum diameter of about 250 to 300 microns. Preferably the bundle has a diameter more in the range of 50-75 or 100 microns. One of the fibers 4 near the center of the bundle is selected for coupling to the excitation light source or laser diode, while the rest of the fibers 5 form a branch to carry the fluorescence to the detector. Experimental results have shown that this embodiment provides excellent coupling efficiency at low cost.

In another embodiment, a dicroic combiner/splitter box is used to combine the optical paths. The light is collimated and then combined using a wavelength selective dicroic that reflects light of the excitation wavelength but transmits the wavelength of light emitted by the pH indicator. This dicroic combiner is placed 45° to the two light paths and this aligns the two optical paths, reflecting the excitation into the sensor fiber while allowing the emission to pass through the dicroic combiner and into the fiber leading to the detector. Experimental results show this to be an effective embodiment, although somewhat lower in performance than disclosed alternatives.

Though two embodiments are herein described, the invention is not so limited to the embodiments set forth herein. It is anticipated that other suitable embodiments may be apparent to those skilled in the art with the benefit of this disclosure.

### 8. Detection of the Resulting Fluorescence and/or Phosphorescence Signal

Small phosphorescence lifetime instruments have been built that measure about 6×7×2.0 inches (or about 12 cm×10 cm×4 cm) and is light weight, but this will be decreased to less than 3×4×1.5 inches. The circuit board is currently single sided and also has substantial open space for prototyping, but is also available as a multilayer board, wherein components are mounted on both the top and bottom of the board. The laser diode driver, is added either on board or as a daughter board, and a fiber coupled laser diode.

Photodetection devices are well understood and readily used in the art, and further discussion of the phosphorometer photodetector (PD) is not believed to be necessary for the practice of the present invention by the skilled practitioner. All are herein included, e.g., photomultipliers, photodiodes, including silicon PIN photodiodes with a built-in preamp, and avalanche photodiodes (APD), including silicon APD. With respect to partial pressure oxygen measurement, a sine wave signal of the desired frequency can be generated by a digital signal processor (DSP) system for digitizing and quantifying a phosphorescence signal, including determination of a phase shift relative to the light output of the LED and of the phosphorescence signal magnitude.

The fluorometer or phosphorometer photodetector output is amplified to provide a signal of optimal voltage for digitizing by the analog-to-digital converter (ADC). A photodiode with an internal amplifier is selected for the optimal light sensitive surface area and lowest noise level. For example, the Hamamatsu Corporation HC120 analog photomultiplier tube assembly with an R3823 photomultiplier has an appropriate surface area (more than 5 mm²) and excellent photosensitivity, in the 500 v to 900 nm wavelength range, as manufactured by Hamamatsu Photonics, KK of Hamamatsu, Japan.

In one embodiment of the present invention, the emitted light is filtered and detected with an avalanche photodiode. The output of the detector is amplified and passed to a 16 bit (or greater) ADC, e.g., but not limited to, a Delta-Sigma digitizer operating at 48 or 96 kHz. This signal is used to control the current in the LED driving circuit. The LED driver circuit is preferably designed to provide greater than 90% modulation of light output by adding a DC signal to the sinusoidal signal, such that the minimum current is just above the threshold for light emission. Above this threshold light output is nearly a linear function of the current through the LED.

The signal from the photodetector may be further amplified with an AC-coupled operational amplifier. In an embodiment using a continuously modulated light source, a phase lock amplifier system may be used to determine the decay (phase shift) between the excitation and fluorescence and thereby the phosphorescence decay constant (“lifetime”). The measurements could be repeated as rapidly (up to 40 to 100 times per second) or as slowly (once every few minutes) as needed. The present invention thus provides stable measurements of oxygen pressure over extended periods of time. The quality of the phase detection depends on the reduction of noise level in the photodiode output signal.

The signal from the photodetector may be further amplified with an AC-coupled operational amplifier. In an embodiment using a continuously modulated light source, a phase lock amplifier system could be used to determine the decay (phase shift) between the excitation and fluorescence and thereby the phosphorescence decay constant (“lifetime”). The measurements could be repeated as rapidly (up to 40 to 100 times per second) or as slowly (once every few minutes) as needed. The present invention thus provides stable measurements of oxygen pressure over extended periods of time. The quality of the phase detection depends on the reduction of noise level in the photodiode output signal.

### 9. Measuring and Recording Data

The measured pH fluorescence or values of oxygen pressure may be presented in any form the user desires, for example, after amplification, the output signal is delivered to the analog multiplexer and then input into the analog-to-digital converter (ADC) for digitizing. Data collection from the digitizer is synchronized with readings of the tabulated values into the digital-to-analog converter (D/A unit) providing the driving current for the light source. Data collection is always begun at the same point in the table of values controlling the light output, e.g., the LED light output.

The respective systems also comprise an instrument with a central processor for measuring the fluorescence and/or phosphorescence from the fiber optic sensor chamber. As described above, small and efficient instruments are the preferred embodiments for the system. The instrument for measuring the fluorescence and/or phosphorescence from the sensor in the chamber is of similar design to those in common use, except that it is intended to be small, with a low power requirement, and capable of use under difficult conditions. The instrument has a fiber coupled monochromatic light source(s) that sends light into one branch of the combiner. This light goes to a port for coupling, into which the optical fiber of the fiber optic sensor chamber can be connected, preferably using a quick connect device. The port is the common end of the branched light guide such that the collected fluorescence is conducted through the second branch to a detector, preferably a photomultiplier, avalanche photodiode or photodiode or other device that converts the light signal into electrical current or voltage

As embodied, the digitized fluorescence and/or phosphorescence data is transferred to a specific file in memory, preferably a 1024×32 bit block of memory. Further data sets (a total of m data sets) are added to the same memory area, always beginning at the same point. Because the collected data are “locked” to the table of values being used to control the excitation light, only signals of exactly the same frequencies as those used to generate the excitation signal are summed positively. All other signals (and noise) are summed destructively, and their amplitudes decrease as the number of scans (m) increases. Noise amplitude, on the other hand, increases only as the square root of the number of scans summed (m½), thus providing increase in signal-to-noise ratio. In an exemplified configuration, 20 data sets are summed. Assuming that each data set is approximately 20 msec long (1024 points at 48 kHz), summing the 20 sets would require less than 0.5 seconds.

Measuring the Emitted Phosphorescence: Measurements of the present invention are readily adapted for low levels of oxygen, such as would be found in hypoxic tissue. The present optical method is not dependent on sample path length or light scattering. Measurements of phosphorescence lifetime are independent of the concentration of the phosphor(s) in the sensor solution, so long as the phosphor(s) is present in the solution at a concentration range needed for oxygen measurement. Within the functional concentration range, there is no significant “self-quenching” due to energy transfer from triplet state to ground state phosphor molecules. This is because of the relatively large size and charge of the preferred dendrimer phosphor constructs. Lifetime measurements are independent of changes in absorption and light scattering, as long as the changes do not occur during phosphorescence decay (<1 msec). This makes the method particularly effective in measuring oxygen in sample conditions affected by contaminants, such as blood, dyes or other colored components within the tissue.

Based upon the principle that the beam of excitation light passed through the environment will equally excite the phosphors in the sensor solution at all levels, and because the phosphorescence lifetime increases as the oxygen concentration in its immediate environment decreases, the calculated values are necessarily greater for points of lower oxygen concentration. Phosphorescence may be measured by any available means in accordance with the present invention.

In general, there are two conventional methods for measuring phosphorescence lifetime (or decay time) are (i) the “pulse method” in the time domain, and (ii) the “phase method” in the frequency domain. The exemplified embodiments of the invention are based upon applications of the phase method, although both may be used, and in the art are considered to be equally effective.

In the pulse method embodiment, the phosphor is excited by a short pulse of light and the resulting phosphorescence emission in the longer wavelength is an exponentially decaying function with a measurable rate of decline. The pulse method is used in the majority of existing instruments for oxygen measurement.

By comparison, in the preferred phase method embodiment of the present invention, the phosphor solution is excited with modulated light, with absorbed light being re-emitted as phosphorescence after a certain delay period. As a result, phosphorescent emission is also modulated with the same frequency, but delayed in time (phase shifted) with respect to the excitation wave. The resulting phase shift, found experimentally, is used to calculate the emitted phosphorescence lifetime.

The phase method embodiment is preferably used herein, because frequency lock amplification can be advantageously used to greatly increase sensitivity. It also allows use of much lower intensity and more durable light sources, which can be of significant advantage with respect to long term stability and reliability of the instruments. Interference from ambient light is greatly decreased by this method, since only signals with the same modulation frequency as the excitation light are amplified, which largely eliminates interference by other ambient light sources.

The phosphorescence lifetime measurements and calculations may be fully automated in certain embodiments of the invention. The values of the phosphorescence intensities and lifetimes may also be recorded or tabulated for later analysis, and the measurements may be repeated as often as necessary until the desired endpoint is reached. The time point at which each data point is measured is recorded, from which the oxygen concentration can be calculated. Measurement of the phosphorescence lifetime is extremely reproducible from instrument to instrument, due partly to the absolute calibration and partly to the nature of the lifetime measurements.

In practice of an embodied method of the invention, following excitation, phosphorescence is collected, optionally passed through appropriate filters, and carried to the recording apparatus of the present invention to obtain the phosphorescence lifetime measurements and calculated oxygen pressure using the relationships disclosed below, e.g., Eq. 1. See. FIG. 3. Quenching of phosphorescence lifetime by oxygen is determined by the frequency of collisions between the excited triplet state molecules and oxygen. Thus the measured phosphorescence lifetime is converted to oxygen pressure according to the Stern-Volmer relationship, Equation 10, above.

In the phase approach, the mathematical relationship between phase shift and phosphorescence lifetime can be described as tan φ=2πft, where φ=phase difference (phase shift) between excitation and emission sine waves at the modulation frequency, f, and t=lifetime of phosphorescent decay. It can be shown that for a given signal-to-noise ratio, the lowest error in the estimation of the phosphorescence lifetime is obtained when the phase shift is about 26°. Engineering principles establish that values from 5 to 40° may be used, but in the present readings, the most accurate values are near 28° phase shift. The difference between 26° and 28° phase shift is not critical for these calculations. Therefore, it follows from the Stern-Volmer relationship and the diffusion equation that to maintain the phase shift of about 26° for all oxygen concentrations in the range, it is necessary to be able to vary the modulation frequencies from 20 Hz to 20,000 Hz. However, it is preferred that modulation frequencies be controlled from 100 Hz to 20,000 Hz, and instrumentation may be employed which can measure phosphorescence lifetime of a given fixed frequency and/or at a first estimate optimal frequency for a given value of the phase shift)(35.5°), and to then proceed with actual lifetime measurements. To ensure oxygen measurements are accurate to air saturation and above (lifetimes as short as <15 μsec), the phosphorescence signal is preferably sampled (digitized) at 48 kHz or greater.

The digital signals are processed to extract the signal strength (magnitude) and phase relative to the excitation light. Calculations of the phosphorescent lifetime and oxygen pressure will follow the above-described procedures. The measured oxygen pressures are closely correlated with the oxygen pressure in the capillary bed in the tissue and provide a measure of the integrated function of the performance of the cardio-pulmonary system.

Measuring Fluorescence. The excitation light is switched between the wavelength absorbed primarily by the protonated form of the dye and the unprotonated form of the dye. The fluorescent signals from the two different measuring lights (different excitation wavelengths of light) are digitized and the digital signals used to calculate the ratio of the fluorescence intensities. This ratio is a direct measure of the ratio of the protonated and unprotonated forms of the fluorescent dye. With this ratio and the pKa for the dye, the pH of the buffer solution (e.g., bicarbonate) can be accurately calculated. Since the concentration of bicarbonate is known, the CO₂ pressure in the buffer can be calculated.

### 10. Alternative Embodiments

In another embodiment of the invention, at least a portion of the optical fiber(s) at the point where the sensor chamber B is operably connected within the gas-permeable layer seal 1 and 6, is faceted, etched, or configured to have a plurality of scratches, depressions, grooves, pitting or otherwise, holes and the like. As a result, emitted phosphorescence and/or fluorescence has an increased probability of being collected by the fiber for return to the detector. In effect, the phosphor and/or fluorophor solution in the chamber, as a result of the grooves, etching, etc. becomes a part of the optical fiber. Each of the plurality of grooves is not more than 20% of the fiber diameter in depth, to allow for sufficient fiber strength, while at the same time allowing for the phosphor solution to penetrate well into the fiber. Such etching may substantially increase the probability of phosphorescence and/or fluorescence entering the fiber within the collection angle.

In an alternative embodiment of the oxygen sensor, also using sensor molecules that absorb and emit in the near infrared, oxygen sensors are used that are encapsulated in physiologically-acceptable polyethylene glycol (PEG). The PEG encapsulation has recently been approved by the FDA for human use, although before clinical use, the selected phosphor would also have to be approved. Thus, the PEG encapsulation replaces the gas-permeable film over the analyte, and when combined with the placing the light source and detector on the skin surface, would permit the PEG encapsulated sensor to be directly injected into the patient's tissue, such as muscle, preferably at depths of less than 1 cm or not more than 2 cm to make measurements easier. Such intramuscular injections of PEG encapsulated phosphors have been shown to distribute into the interstitial space within the tissue and remain there for several hours without washing away in a tissue environment, thus accurately reporting the oxygen pressure in the tissue. Only a few micrograms of the injected PEG-coated sensor would be required, and then the analyte could be excited from the skin surface and detected as describe for the wireless, surface system above. Such a system, once approved would offer a simple, effective, inexpensive and highly portable method for rapidly measuring tissue oxygen, and may eventually become the preferred method of choice, particularly for emergency purposes.

To illustrate the effectiveness of the present oxygen monitoring device, several clinical applications are provided in the following examples, but while exemplary, they are not intended to in any way limit the breadth of the invention which is, in fact, limited only by the breadth of the claims defining the invention.

### EXAMPLES

There are several clinical situations in which the ability to monitor delivered oxygen to specific tissues on a real time continuous basis would help improve patient care. These include, without limitation, patients who: 1) have undergone abdominal surgery for ischemic bowel, 2) have had a surgical muscle flap created, especially a free flap, and 3) those who have a need for cardiopulmonary resuscitation, since prior art methods for monitoring oxygen levels in the patient in each situation is inadequate.

### Example 1

### Monitoring of an Ischemic Bowel

Pediatric and adult patients can develop conditions, such as volvulus, necrotizing enterocolitis and strangulation of the intestine due to an adhesion. These cause regional ischemia of the intestine requiring an exploratory laparotomy and possible resection. Often there are areas of the intestine that are transition zones with potential viability. To help preserve as much of the intestine as possible a second (or third) surgical look may be required to assess these areas. Further, there is no way to judge the outcome of therapies to improve intestinal viability until it is reassessed visually. Computerized tomography is of limited use and usually cannot distinguish viable from non-viable tissue, except at the irreversible extreme. Plain X-rays are also only useful at the extreme, when perforation has occurred due to tissue necrosis.

Solution using present invention: Following the initial laparotomy, the surgeon can, in accordance with the present invention, place an oximeter catheter, within or attached to a surgical drain(s) in the area of concern. Also, one may have multiple individual fiber optic bundles monitoring areas spaced along the length of the catheter. Further, a lattice could be created with the catheter material that could monitor the two-dimensional areas As further set forth in U.S. Pat. No. 6,274,086, herein incorporated by reference, two- and three-dimensional oxygen imaging of tissue is accomplished by measuring phosphorescence emission of the oxygen-quenchable compounds in an apparatus comprising a matrix of light guides and/or phosphorescence detectors to allow precise and sequential introduction of pulses of excitation light from a plurality of sites in the matrix. As a result, if ischemia is detected, the clinician can rapidly consider strategies to improve perfusion while the bowel is still viable and accessible, before necrosis makes repair impossible.

### Example 2

### Monitoring of a Muscle Flap

As part of restorative surgery to fill in a space created by re-section of diseased tissue or loss from trauma, surgeons often mobilize muscle from one area and transfer it to another. This muscle may still have its native vascular supply intact, or it may be completely disconnected, in which case it is reattached to another vascular supply (free flap). Such surgery is often complicated by flap failure due to an inadequate vascular supply, and unfortunately, it is often difficult to monitor the integrity of the flap because it is subcutaneous. Doppler ultrasound may be used, but it can only determine whether a pulse can be detected in or near the tissue.

Solution using the present invention: An oximeter catheter of the present invention could be inserted along the body of the flap or inserted into the body of the muscle, and the integrity of the muscle can then be monitored at various points on the flap while in situ. This could be easily removed along with surgical drains once the condition of the flap has been insured, or at the time of a second operation. As a result, if ischemia is detected, the clinician can consider strategies to improve perfusion while the flap is still viable, and as above, before necrosis has caused irreversible damage.

### Example 3

### Monitoring Cardiopulmonary Resuscitation

The American Heart Association has established guidelines for providing cardiopulmonary resuscitation (CPR) to victims of cardiac or respiratory arrest. One of the difficulties in providing this potentially life saving care, is the inability to monitor in real time, the adequacy of chest compressions and the delivery of oxygen into the tissues of the patient. In an intensive care unit a patient may have an arterial line already established, permitting medical practitioners to periodically sample the patient's blood to monitor progress. However, before the patient reaches the ICU, arterial lines are not used because they take time and expertise to establish, making them impractical to use in an acute situation. As a result critical measurements of tissue oxygen are not possible.

Solution using the present invention: At initiation of CPR, one could insert the oximeter catheter into a deltoid, masseter or other muscle as a surrogate for cerebral perfusion and/or oxygenation. One could then monitor delivered oxygen to those tissues on a continuous basis during CPR, and thus monitor the quality of the resuscitation in critical tissues. In addition, because tissue temperature and gas levels can be significantly different than temperature and gas levels in the circulating blood, the real-time measurement of tissue parameters is important because, as above, progressive decrease in pO₂ and increase in pCO₂ provides an accurate measure of progression from healthy conditions toward shock and multi-organ failure.

Brief, but repeated, intermittent severe episodes of asphyxia, as observed with central or obstructive apnea in the human neonate, infant or child may also result in clinically important deficits in neurophysiologic function. The brain injury caused by repeated, intermittent, severe hypoxic-ischemic insults, is often more pronounced than that caused by the same period of continuous hypoxia-ischemia. For example, sleep disordered breathing, relevant during intermittent, severe apnea and resuscitation may result from a variety of causes, including: 1) Neurologic: idiopathic central apnea, epilepsy; prematurity 2) Pharmacologic: antiepileptics, prostaglandins, sedative, anesthetic, analgesic medications; 3) Toxin related: carbon monoxide poisoning; 4) Gastro-esophageal reflux (aspiration causing laryngospasm and obstructive apnea 5) Anatomic: laryngomalacia, 6) Infection: sepsis, meningitis, infant botulism, bronchiolitis (Respiratory Syncitial Virus) 7) Child abuse: including Munchausen syndrome by proxy, and physical abuse; and 8) Congenital: central hypoventilation syndrome, dysautonomic syndromes. Use of the present invention will lead to an understanding and control of how differences in oxygen enrichment of resuscitation gas concentration affect brain metabolism and cellular neuropathologic processes leading to cell recovery or cell death is critical to improving outcomes following brief, intermittent, severe episodes of asphyxia, as well as injury or disease.

With such understanding and control, the present invention will also permit mild hypothermia to be applied beneficially in certain circumstances, such as before, during or immediately following global ischemic insults, include decreased cerebral metabolism, anti-inflammatory effects, decreased glutamate concentrations, decreased generation of free radicals and lipid peroxidation, decreased heat shock protein response and kinase activation.

Moreover in controlled situations, such as through the use of the present invention, hypothermia has been shown to have selective anti-inflammatory effects, decreasing expression of NF-κB and secretion of interleukin-8 by cerebral endothelial cells thereby inhibiting leukocyte recruitment to the cerebral microcirculation. Recent research has demonstrated that mild hypothermia implemented within 1-6 hours following the ischemia for durations of 12-48 hours, improved both histologic and functional outcomes. Conversely, in certain circumstances, mild hypothermia protects against neuronal loss in selectively vulnerable brain hippocampus (CA1) and improves neurobehavioral outcome following five minutes of global (two vessel occlusion) cerebral ischemia, which in test animals has been sustained for at least 6 months following the injury. In the clinical environments, mild systemic or selective brain hypothermia (32-34° C.) has effectively treated selected infants with global birth asphyxia and related neonatal encephalopathy, when the hypoxic-ischemic etiology is even a sustained insult.

In any of the above described embodiments, the system may further comprise a temperature sensor circuit designed for measuring the temperature at the site of the insertion. Although temperature measurements are not novel, temperature measurements are sensitive to alterations in peripheral blood flow. It is expected that increasing pCO₂ due to decreased blood flow or inappropriate blood flow is accompanied by a decrease in tissue temperature. Therefore, the system is designed to provide a temperature measurement complimentary to the oxygen pressure and/or pCO₂ measurement with respect to the clinical/physiological measurements of the biochemistry and physiology of animals and humans. In addition, the temperature reading allows for correction of any temperature dependence of the calibration of the oxygen sensor and the pK of the pH sensor dye.

As further embodied, the system also comprises a display for visual presentation of the oxygen pressure, pCO₂, and/or temperature data. Wireless transmission of oxygen pressure, pCO₂, and/or temperature data to a central monitor allows physicians to remotely monitor the patient's real-time status. Advantageously, as embodied, each system disclosed herein is of such a compact nature that the sensor may be inserted into the patient's tissue for continued monitoring, and the external portion of the system may be comfortably taped to the body of the patient, particularly useful when the system is battery powered

The measurements and calculations may be fully automated in certain embodiments of the invention. The values of the phosphorescence intensities may also be recorded or tabulated for later analysis, and the measurements may be repeated as often as necessary until the desired endpoint is reached. The time point at which each data point is measured is recorded, from which the pCO₂ and/or oxygen pressure/concentration can be calculated. Measurements are extremely reproducible from instrument to instrument, due partly to the absolute calibration and partly due to the nature of the measurements.

The disclosures of each patent, patent application and publication cited or described in this document are hereby incorporated herein by reference, in their entirety. However, the disclosed dates of publication may be different from the actual publication dates, which may need to be independently confirmed. No reference identified herein is to be construed as an admission that the present invention is not entitled to antedate such publication by virtue of prior invention.

While the foregoing specification has been described with regard to certain preferred embodiments, and many details have been set forth for the purpose of illustration, it will be apparent to those skilled in the art, that without departing from the spirit and scope of the invention, the invention may be subject to various modifications and additional embodiments, and that certain of the details described herein can be varied considerably without departing from the basic principles of the invention. Such modifications and additional embodiments are also intended to fall within the scope and spirit of the invention appended claims.

What is claimed is:

1. An tissue-insertable, in vivo system for real-time measurement of tissue pCO₂ of an animal or human patient, the system comprising:
a fiber optic sensor chamber forming a probe having the wall-strength to withstand external tissue pressure;
a highly fluorescent, aqueous, buffered, pH-sensitive fluorophor, sealed within the fiber optic sensor chamber;
an excitation light source for activating the fluorescence of the fluorophor; and
an instrument for measuring and reporting fluorescence from the activated fluorophor in the fiber optic sensor chamber in place within the tissue of the patient, from which level(s) of pCO₂ are calculated.

2. The system of claim 1, wherein the fluorophor comprises a pH sensitive porphyrin-based dye.

3. The system of claim 1, wherein the pH buffer is bicarbonate buffer, sealed within the fiber optic sensor chamber.

4. The system of claim 1, further comprising optical fibers to 1) transport the excitation light to the fluorophor, and 2) to transport the fluorescence from the fluorophor following excitation to the measuring and reporting instrument.

5. The system of claim 3, further comprising wireless connections to 1) transport the excitation light to the fluorophor, and 2) to transport the fluorescence from the fluorophor following excitation to the measuring and reporting instrument.

6. The system of claim 3, further comprising a combiner for coupling the excitation light source to the optical fiber of the fiber optic sensor chamber.

7. The system of claim 6, further comprising an amplifier to amplify the fluorescence signals.

8. The system of claim 1, further comprising a central processor for calculating and reporting fluorescence measurements.

9. The method of claim 1, further comprising a temperature sensor circuit for measuring temperature at the site of insertion.

10. A tissue-insertable probe device containing therein a fluororphor analyte within a sealed sensor chamber, wherein the fluorophor operably responds to pH levels in the surrounding tissue, thereby providing calculated pCO₂ levels in the tissue.

11. The probe of claim 10, further comprising one or more aligned optic fibers, each having two opposing ends, and each of which is operably connected and sealed at the distal end to the probe, thereby forming an operably-linked light guide for collecting emitted fluorescence from the fluorophor at less than the numerical aperture of the light guide; wherein at the proximal end, at least one first fiber is externally, operably-connected to the light source to transmit excitation light to the fluorophor within the sealed sensor chamber, and wherein at least one second fiber is externally connected to the detecting device to collect and transmit emitted fluorescence from the fluorophor to the detector device.

12. A method for making real-time, in vivo measurement of tissue pCO₂ in the animal or human patient, the method comprising:
inserting into the tissue of a patient the probe containing the sealed fiber optic sensor chamber containing the buffered, pH sensitive fluorophor into the tissue;
activating the excitation light source to excite the fluorophor;
measuring the fluorescence from the excited fluorophor; and
calculating pCO₂ from the pH measurement.

13. The method of claim 12, wherein the fluorophor comprises a pH sensitive porphyrin-based dye.

14. The method of claim 12, wherein the pH buffer is bicarbonate buffer.

15. The method of claim 12, further comprising connecting optical fibers to 1) transport the excitation light to the fluorophor within the sensor chamber, and 2) to transport the fluorescence from the fluorophor following excitation to the measuring and reporting instrument.

16. The method of claim 15, further comprising connecting a combiner for coupling the excitation light source to the optical fiber of the fiber optic sensor chamber.

17. The method of claim 16, further comprising connecting an amplifier to amplify the fluorescence signals.

18. The method of claim 12, further comprising connecting a central processor for calculating and reporting fluorescence measurements.

19. The method of claim 12, further comprising connecting a temperature sensor circuit for measuring temperature at the site of insertion, and measuring temperature.

20. The system of claim 1, further comprising in the system an element for real-time measurement of tissue oxygen lifetime in the tissue, said system comprising:
a oxygen-quenchable phosphor solution within the probe, wherein refractive index of the phosphor solution is higher than that of the surrounding gas-permeable layer;
an excitation light source for activating the phosphorescence of the phosphor; and
an instrument for measuring and reporting phosphorescence from the activated phosphor from within the tissue of the patient.

21. The system of claim 20, wherein the phosphor comprises an aqueously soluble oxygen quenching, dendrimeric metalloporphyrin sensor, which is capable of phosphorescence, and having the formula: 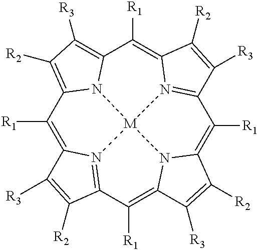
wherein: R1 is substituted or unsubstituted aryl; R2 and R3 are independently hydrogen or are linked together to form substituted or unsubstituted aryl; and M is H2 or a metal.

22. The probe of claim 10 further comprising:
a phosphor solution within the probe, wherein refractive index of the phosphor solution is higher than that of the surrounding gas-permeable layer;
an excitation light source for activating the phosphorescence of the phosphor; and
an instrument for measuring and reporting phosphorescence from the activated phosphor from within the tissue of the patient.

23. The probe of claim 22, wherein the phosphor comprises an aqueously soluble oxygen quenching, dendrimeric metalloporphyrin sensor, which is capable of phosphorescence, and having the formula: 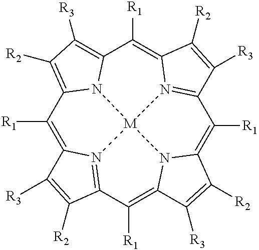
wherein: R1 is substituted or unsubstituted aryl; R2 and R3 are independently hydrogen or are linked together to form substituted or unsubstituted aryl; and M is H2 or a metal.

24. A method of using the system of claim 12 for also making real-time, in vivo measurement of tissue pO₂ and oxygen pressure in the animal or human patient, the method comprising:
adding an oxygen quenchable phosphor solution into the probe containing the sealed fiber optic sensor chamber containing the buffered, pH sensitive fluorophor, or adding a second sealed fiber optic sensor chamber containing the oxygen quenchable phosphor solution into the probe;
inserting the probe into the tissue;
activating the excitation light source to activate the phosphor as well as the fluorophor and the phosphor;
measuring and reporting the phosphorescence from the excited phosphor for real-time measurement of tissue oxygen lifetime in the tissue as well as the fluorescence from the excited fluorophor.

25. The method of claim 24, wherein the phosphor comprises an aqueously soluble oxygen quenching, dendrimeric metalloporphyrin sensor, which is capable of phosphorescence, and having the formula: 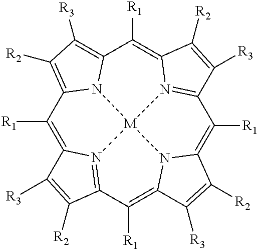
wherein: R1 is substituted or unsubstituted aryl; R2 and R3 are independently hydrogen or are linked together to form substituted or unsubstituted aryl; and M is H2 or a metal.

26. The method of claim 25, further comprising one or more of the additional steps consisting of connecting optical fibers to 1) transport the excitation light to the phosphor within the sensor chamber, and 2) to transport the phosphorescence from the phosphor following excitation to the measuring and reporting instrument; connecting a combiner for coupling the excitation light source to the optical fiber of the fiber optic sensor chamber; connecting an amplifier to amplify the phosphorescence signals; and connecting a central processor for calculating and reporting phosphorescence measurements.

27. The method of claim 12, further comprising monitoring oxygen supplied to the patient's ischemic bowel, to the patient's surgically transplanted muscle flap or to the patient's tissue during cardiopulmonary resuscitation.
